(19) United States
(12) Patent Application Publication
(10) Pub. No.: US 2002/0155467 A1
(43) Pub. Date: Oct. 24, 2002

(54) Method for the determination of at least one functional polymorphism in the nucleotide sequence of a preselected candidate gene and its applications

(76) Inventor: Jean-Louis Escary, Le Chesnay (FR)

Correspondence Address: Mark A. Hofer, Brown, Rudnick, Freed & Gesmer, One Financial Center, Boston, MA, 02111, US

(21) Appl. No.: 10/010,749

(22) Filed: Dec. 6, 2001

(30) Foreign Application Priority Data
Dec. 6, 2000 (FR) 0015838

(51) Int. Cl. C12Q 1/68

(52) U.S. Cl. 435/6

(57) ABSTRACT

The present invention concerns a method for determining at least one functional SNP in a gene, comprising preselecting a candidate gene, providing a sample population comprising a significant number of individuals chosen substantially at random from the general population, isolating from each individual of the sample population at least one fragment of the nucleotide sequence of the preselected candidate gene, identifying at least one SNP in at least one fragment and determining the functionality of said SNP(s). The present invention also concerns applications of this method.

### RELATED APPLICATIONS

This Application claims the benefit of pending French Application FR 0015838, in its entirety, filed on Dec. 6, 2000, which is also incorporated herein as reference.

### BACKGROUND OF THE INVENTION

1. Field of the Invention

The present invention relates to the determination of one or more polymorphisms in the nucleotide sequence of a preselected candidate gene and its applications.

2. Background Art

Recognition of the importance of polymorphisms in the human genome increases daily, especially with regard to research into the causes of certain diseases or particular sensitivities and for research into medications which may directly effect specific genomic targets.

There is a genetic and an environmental contribution to the manifestation of common diseases in humans and to the resistance of certain individuals to these same diseases. Sensitivity to or genetic resistance to common diseases will hereafter be referred to as “traits.”

With regard to genetic contribution, the molecular genetics community recognizes that: (i) the number of genes that contribute to these exceeds one (polygenic origin of traits), and (ii) the majority of these traits are attributable to variations in expression or function of the genes. In addition, the majority of these variations are suspected to be variations of a base pair or Single Nucleotide Polymorphisms (SNPs). SNPs represent on average a total of 0.1% of the entire human genome sequence or nearly 3 million base pairs.

The characterization of functional SNPs will reveal the presence of candidate genes that predispose individuals to common diseases. Without being restricted to this characterization, many believe SNP research will enable development of specific therapeutic molecules for common diseases. These specific therapeutics may enable correction of the protein structures encoded by the candidate genes comprising functional SNPs.

Characterization of functional SNPs will also uncover relationships between the mutant alleles and the resistance of certain individuals to particular common diseases. Again, without wishing to be limited to this theory, the resulting therapeutic molecules should protect individuals from their deleterious alleles, possibly by altering the structure of the corresponding carrier proteins.

In short, these therapeutic molecules and diagnostic/prognostic kits should be the keys for prevention and treatment of common diseases.

Current efforts of post-genomic research focus on functional SNPs that exhibit relationships between one or more mutant alleles and one of the two traits, sensitivity or resistance to common diseases in the population. Accordingly, this research typically entails genotyping analysis of samples from persons preselected for one of the two traits in order to search SNPs, followed by statistical analyses of associations between certain alleles comprising said SNPs and the trait(s) of interest.

Typically, the individuals for whom the genotype is determined are selected based on specific phenotypic criteria such as medical, clinical, epidemiological, physiological and biological criteria, all of which measure the degree of sensitivity or resistance of these individuals to particular common diseases.

Up to now, therefore the research into variations in nucleic sequences, especially those called SNPs, that is, those concerning one nucleotide, has been carried out either systematically via sequencing of the human genome or by sequencing the genomic DNA of individuals who, for example, have a particular sensitivity or resistance.

The most common method consists of investigating a direct relationship between a mutant allele comprising a functional or nonfunctional SNP and one of the two traits of common diseases.

This is broadly accomplished through four steps:

(i) identifying the SNPs in (a) a sample including patients and/or individuals displaying a resistant phenotype and (b) a sample from individuals known as controls (individuals presenting normal phenotypic data regarding the trait(s) studied. The SNPs are researched on the genome in order to determine either an association or a genetic linkage between one or more regions of the genome and the trait(s) at issue (“Genomescan” approach).

(ii) genotyping alleles comprising SNPs identified in step (i) from the patients and/or resistant individuals, and control individuals, followed by statistical analysis of the associations or genetic linkage between genotype allele(s) and the trait(s) at issue.

(iii) analyzing genotyping data as follows: statistical calculations are used to estimate the degree of reliability for the genetic association between the higher frequency of one or more allele(s) in individuals displaying the selected trait(s) versus control individuals. The genetic associations confirmed by the statistical calculation between one or more functional SNP(s) and the selected trait(s) thereby reveal a relationship between the variability of expression or function of the carrier gene(s) and protein(s) and the trait. This information enables evaluation of current therapeutic targets with regard to the mutant alleles studied. Using this method, the recent decoding of the human genome sequence and the sequencing of numerous new genes on the genome will enable the identification of numerous new therapeutic targets for the prevention and treatment of common diseases.

(iv) confirming the status of the therapeutic targets for certain alleles comprising functional SNPs and identified as genetically associated with the trait of interest. This is done by developing biological tests that establish the relationship between the allele and the trait by a modeling method. For example, it may be shown that the mutant allele comprising a SNP found in the promoter region of the candidate gene has an effect on the expression of the gene, or even that the mutant allele comprising a functional SNP found in the coding sequence of a candidate gene has an effect on the structure of the protein coded by the gene, and even more on the structure of the active domains of this protein, showing a clear effect of the mutant allele on the activity of said protein and therefore of the gene. This biological information is indispensable for establishing a functional link between the genetic study of the trait and the medical, clinical, physiological or biological data collected, and for selecting the sick people or resistant people according to the trait studied. From this functional link established between certain alleles and the trait studied, and the characterization of the biological impact of the allele concerned on the expression or the function of the gene or protein studied, diagnostic/prognostic kits and/or new therapeutic molecule(s) can be developed.

Gathering a group of individual patients for whom a genetic feature must be determined requires long, expensive and often difficult procedures. Indeed, forming phenotypic groups of interest in which the DNA sequences must be studied is especially difficult because a representative number of persons manifesting a common phenotypic feature must be located, solicited and engaged.

The need exists for a method of reliably discovering the existence of polymorphisms in the human genome without the disadvantages of the above methods, for example expense, lack of certainty, and the requirement of separate study and control groups. Also, systematic sequencing is inefficient since it requires working on sequences without value, and more particularly without therapeutic value.

U.S. Pat. No. 5,795,976 (Oefner et al.; filed on Aug. 8, 1995) relates to a chromatographic method for detecting mutations in nucleic acids isolated from a sample population of 22 individuals having particular phenotypic characteristics linked to the searched mutations for detecting mutations in the human Y chromosome, such as male individuals. This method does not disclose nor suggest the identification of functional SNPs from individuals chosen substantially at random from the population.

WO 01/27857 (Sequenom, filed on Oct. 13, 2000 and published on Apr. 19, 2001) is directed to a method for generating databases of polymorphic genetic markers from individuals having particular phenotypic characteristics, such as healthy individuals and chosen on the basis of precise information such as age, sex, medical data, lifestyle, etc. Furthermore, this method does not permit the identification of the functionality of said polymorphic genetic markers.

### BRIEF SUMMARY OF THE INVENTION

The present invention is directed to a method for determining at least one functional SNP in a gene, comprising the following steps:

a) Preselecting a candidate gene;

b) Providing a sample population comprising a significant number of individuals chosen substantially at random from the general population;

c) Isolating from each individual of the sample population at least one fragment of the nucleotide sequence of the preselected candidate gene;

d) Identifying at least one SNP in at least one fragment isolated in step c); and

e) From the SNP(s) identified in step d), identifying those with functionality.

It is recognized that the impact of the gene pool of a person on his (her) sensitivity or resistance to the appearance and to the development of a disease is due to mutations that change the normal expression and/or the activity of one or more of his (her) genes. The functional SNPs are counted among these mutations. Among them, one or all will therefore form targets for the development of diagnostic, prognostic and therapeutic kits and tools for the prevention and treatment of said diseases.

In this context, the instant invention enables the identification and localization of polymorphisms and especially genomic defects, and it especially presents the following aspects, not limited thereto:

a) The method of the instant invention applies to pre-selected candidate genes which are known to have pleiotropic effects, meaning they are involved in several metabolic pathways and biological processes, increasing the likelihood that they will be useful as therapeutic targets.

b) The method according to the invention is based, in contrast to the prior art, on the identification of functional SNPs in candidate genes in a substantially random population and not on a population selected on medical, clinical, epidemiological, physiological or biological criteria and data, for example. In particular, the method according to the invention enables discovery of functional SNPs in candidate genes in a substantially random population without resorting to the analysis of samples from preselected patients or resistant individuals. This substantially random population preferably takes into account a large number of different human ethnic groups, or subspecies in the case of animals.

One of the aspects of such a sample population is this: given that each individual can be regarded as a potential patient for a given disease and a negative control for another disease, all the common diseases are represented. In contrast to the studies of determination of SNPs based on the comparison of the genomic sequences of a group of patients and a reference group to identify a SNP and to correlate it with a given disease (“classical approach”), the present invention does not present any bias based on the phenotype (the disease, for example) and thus identifies any sequence variation whereas the classical approach only enables detection of sequence variations related to the selected disease and not those related to another disease. Indeed, this is well illustrated by the observation made by the inventor according to whom the sequence variations discovered for a given gene could not be correlated with the studied disease because the experimenter did not select the disease associated with the gene. As an example, the inventor realized this disadvantage, inherent in the classical approach, while he studied SNPs in the GMCSF gene implied in cardiac infarction. Indeed, he could not find any association between the SNPs of this gene, discovered by the classical approach based on patients affected by cardiac infarction, and cardiac infarction. In contrast, surprisingly, a library search showed that one SNP discovered among the patients affected by cardiac infarction was, in fact, associated with asthma, demonstrating the role of this SNP in asthma and not in cardiac infarction.

As a consequence of this aspect of the present invention, the database of SNPs generated by the present method is wider than what one can get from the classical approach based on comparative studies of patients and control groups. In addition, the present method saves a considerable amount of time and money because it permits the development, in one step, of a wide database of SNPs which may be used for any disease, whereas the classical approach would have required as many steps of SNPs identification as investigated diseases.

c) The method according to the invention economizes with any preselection of persons for a particular phenotypic trait, for example a particular sensitivity or resistance to diseases, to discover functional SNPs forming potential diagnostic/prognostic and therapeutic targets on the genome.

The method of the invention therefore saves time, money and energy in the discovery of these potential targets for the development of kits for the prevention and treatment of diseases. This is especially important as it is sometimes very difficult and costly to gain access to a significant number of patients for some particular diseases.

d) Furthermore, the instant method is more reliable for discovering prognostic/diagnostic and therapeutic targets on the genome in comparison to statistical studies of associations or genetic linkages based on genotyping studies of persons sensitive or resistant to the diseases and control persons.

In fact, although measured, the risk is real of discovering an association or a genetic linkage between one or more SNPs and the appearance and/or development of one or more disease(s) while this association or genetic linkage is false in reality (this type of association or genetic linkage is called a false positive association or linkage), this risk cannot be avoided owing to the very statistical nature of the methods of calculation.

In contrast, the present method focuses on relevant SNPs, in regard to common diseases, by describing the development of concrete biological tests which demonstrate the real functional role of certain alleles comprising functional SNPs on the expression or activity of genes and it constitutes a more reliable method to propose potential diagnostic/prognostic and therapeutic targets on the genome.

One aspect of the instant method is to reduce the costs of further clinical trials to establish the involvement of one or more SNPs in a given disease because of the pre-selection of the relevant functional SNPs.

e) The identification of a strong biological effect of these alleles on the expression or the function of the candidate genes or proteins coded by these genes, combined with data from the prior art concerning the functional candidate genes, enables the development of potential therapeutic targets for therapeutic fields (common diseases) for which the candidate genes are suspected in the art of contributing to the disease or the resistance thereto.

Once the SNPs are detected, the identification of the allele(s) genetically associated with the trait(s) of interest and therefore the identification of new therapeutic targets connected with common diseases can be carried out.

The genotyping of individuals chosen substantially at random from the general population for the functional SNPs so identified enables estimation of the allelic frequency of these SNPs in the different human ethnic groups represented in the sample population, which also enables prediction of the impact of the identification for the diagnosis/prognosis or treatment of these different ethnic groups.

An embodiment of the present invention can be encompassed in a two step framework: (i) identification of functional SNPs in a random sample formed from individuals recruited at substantially at random from the general population, and (ii) confirmation of the impact of the mutant allele comprising each of the functional SNPs on the expression or function of the candidate genes or proteins coded by these genes.

Briefly, instead of proceeding systematically as in the prior art using specific individuals (chosen because they are patients or resistant persons) to obtain the genes and to study them, the interest of the present invention lies only in genes known in the state of the art as fulfilling particular functions in a pathology or in a particular biological process, and the genes are studied in a sample comprising individuals chosen substantially at random from the general population, that is, for example, not chosen because they present the characteristic one is trying to study. As no particular data concerning the individuals constituting the sample to be tested is sought, the method of the invention (i) reduces considerably the costs and facilitates the study, (ii) does not limit the study to a group of patients, to a given disease, to a given gender or age, and especially, does not introduce any bias based on the study of a single gene, which instead remains valuable for any disease. The invention inherently eliminates any risk from preselection of the individuals.

### BRIEF SUMMARY OF THE DRAWINGS

FIG. 1 represents the minisequencing that is carried out during genotyping. The nucleotides ddATP surrounded with dotted line are labeled with the fluorophore R110*. The nucleotides ddGTP surrounded by unbroken lines are labeled with the fluorophore Tamra*.

FIG. 2 represents a wild-type profile corresponding to a homozygous individual (top) and a profile corresponding to a heterozygous individual (bottom). The abscissas represent the retention time in minutes. The ordinates represent the intensity in millivolt.

FIG. 3 represents the bioinformatic modeling of the mutated IFNα-2 protein comprising the SNP H34R and the wild type IFNα-2 protein. The black ribbon of FIG. 3 represents the wild type IFNα-2 protein structure. The white ribbon of FIG. 3 represents the mutated IFNα-2 protein structure.

FIG. 4 represents the bioinformatic modeling of the mutated IFNα-2 protein comprising the SNP M148 I and the wild type IFNα-2 protein. The black ribbon of FIG. 4 represents the wild type IFNα-2 protein structure. The white ribbon of FIG. 4 represents the mutated IFNα-2 protein structure.

### DETAILED DESCRIPTION OF THE INVENTION

“Preselected candidate gene”: is designated as a gene where the following is known:

a) all or part of the coding nucleotide sequence and/or the sequence of the protein encoded by this gene, and

b) any medical, clinical, epidemiological, physiological or biological data relative to said gene and which makes it possible to reveal to the experimenter:

a potential or assumed role of the expression of this gene or of the protein(s) encoded by this gene (if it or they exist) in a metabolic or biological pathway,

the biological function of the protein(s) encoded by this gene (if it or they exist), and/or

the involvement of the protein(s) encoded by this gene (if it or they exist) in the appearance of common pathologies and/or diseases or, on the contrary, in a particular resistance to these pathologies and/or diseases in the human population.

The preselection of the candidate gene can be achieved by carrying out a literature search (PubMed or OMIM, for example). The extrapolation of data obtained in models other than the human model (murine, yeast, etc.) is possible but requires the characterization of the human genes/proteins involved in the processes described in these models (for example, by sequence homology or by reconstruction of signaling pathways or metabolic pathways).

The candidate gene is preferably preselected according to data about the gene's suspected role in the appearance of or resistance to a common pathology and/or disease.

The preselection of the candidate gene is based on knowledge or suspicion that the candidate gene plays a role in the appearance of or resistance to at least one pathology and/or disease.

The candidate gene is also preselected by carrying out research in the literature or in databases describing, for example:

the reference wild-type sequence of the gene and the protein(s) encoded by this gene in the human being and/or in any species of the animal kingdom,

the structure of the reference wild-type protein(s) in the human being and/or any species of the animal kingdom,

one or more studies of the structure of the reference wild-type protein(s) encoded by the candidate gene such as crystallography studies,

one or more studies of comparison of the sequence of the reference wild-type gene in the animal kingdom,

one or more experiments of site-directed mutagenesis on the reference wild-type sequence of the candidate gene showing the role of certain amino acids in the function of the protein(s) encoded by the candidate gene,

biological activity tests in vivo in animals or in vitro conducted with human or any other animal cells such as for example tests for proliferation, differentiation, or showing the involvement of the reference wild-type gene or protein in the activation or repression of a metabolic pathway, in particular the regulation of the activity of protein kinases and the nuclear expression of particular genes,

animal models demonstrating the role of the gene or of the protein(s) encoded by the candidate gene in the appearance of a particular pathology (for example transgenic mice), and

epidemiological, medical or clinical data showing an involvement of the gene or the protein(s) encoded by this gene in the appearance of or the resistance to a common disease in the human population.

Among the data on the candidate gene that may be used for the identification and characterization of the functional SNPs, the following is of particular importance:

the knowledge of (i) regulatory sequences of the candidate genes that are responsible for the expression of these genes or protein(s) encoded by these genes and (ii) if they exist, sequences, in the coding sequences, that encode for signal peptides of the proteins encoded by these genes that are responsible for the activity at the proper localization and/or the definitive localization of the protein(s) encoded by these genes,

the knowledge of the three-dimensional structure of the reference wild-type proteins encoded by the reference wild-type sequence of the candidate genes, and

the knowledge of amino acids that have been identified, within these structures, as taking part in the activity of said reference wild-type proteins.

“Nucleotide sequence of a preselected candidate gene”: corresponds generally to a nucleotide sequence which comprises the regulatory nucleotide sequence and the coding nucleotide sequence. The nucleotide sequence of a preselected candidate gene may equally comprise one of the following: CDS sequence, enhancer sequence, silencer sequence, splicing site and mRNA sequence. The nucleotide sequence of a preselected candidate gene is either known entirely or known in part in the prior art and acts as template for the experimenter for the design of fragments of the candidate gene and the PCR (Polymerase Chain Reaction) amplification of these fragments from the genomic DNA of the individuals.

This nucleotide sequence may also be called the wild-type nucleotide sequence of a preselected candidate gene. This corresponds to the assumed wild-type allele known in the prior art, which is used as a reference.

The protein encoded by the nucleotide sequence of a preselected candidate gene may be known in the prior art or determined by the experimenter from the nucleotide sequence of the preselected candidate gene by methods known in the prior art.

It is also acknowledged that in the case where the nucleotide sequence of the preselected candidate gene is not entirely known in the prior art, the person skilled in the art can determine the missing part and integrate it. To do so, the person skilled in the art may apply his or her own technological resources including, for example, cloning and sequencing of all the regulatory and coding sequences of the candidate gene using complete or partial sequencing of a genomic clone containing all or part of the sequence of the candidate gene.

“General population” corresponds to the world population of individuals as a whole.

“Sample population” corresponds to a group of individuals chosen substantially at random from the general population. An individual may be an animal, such as human, a plant, a virus, a bacteria, a fungi and/or a yeast. Human individuals may be chosen according to their belonging to a specific ethnic population, such as, for example, African American, Southwestern American Indian, South American (Andes), Caribbean, North American Caucasian, Iberian, Italian, Mexican, Chinese, Japanese, Greek, Indo-Pakistani, Middle-Eastern, Pacific Islander, South Asian and South American, in order to constitute a representative sample of the world population or be chosen among one or more ethnic populations. The sample population may also be called the random population.

“Substantially at random”, when applied to the sample population, means that, in the sense of the present invention, the individuals are chosen without regard to the phenotypic and/or genotypic characteristics that are or may be linked to the preselected candidate gene in their genome.

Preferably, when the individuals are chosen substantially at random no attention is paid to the genotypic and phenotypic criteria including for example, the collection of medical, clinical, epidemiological, physiological or biological data.

“Significant number of individuals” is understood to be a number of individuals and therefore of genes studied, for example, greater than 100, especially greater than 150, preferably greater than 200, and very particularly greater than 250.

“Polynucleotide” is defined as a polyribonucleotide or a polydeoxyribonucleotide that can be a modified or non-modified DNA or RNA.

The term polynucleotide includes, for example, single stranded or double stranded DNA, DNA composed of a mixture of one or several single stranded region(s) and of one or several double stranded region(s), single stranded or double stranded RNA, or RNA composed of a mixture of one or several single stranded region(s) and of one or several double stranded region(s). The term polynucleotide may also include RNA and/or DNA including one or several triple stranded regions. By polynucleotide is equally understood DNA and/or RNA containing one or several bases modified for reasons of stability or for other reasons. By modified base is understood, for example, the unusual bases such as inosine.

“Polypeptide” is defined as a peptide, an oligopeptide, an oligomer or a protein comprising at least two amino acids joined to each other by a normal or modified peptide bond, such as in the cases of the isosteric peptides, for example.

A polypeptide can be composed of amino acids other than the 20 amino acids defined by the genetic code. A polypeptide can equally be composed of amino acids modified by natural processes, such as post-translational maturation processes, or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends.

A polypeptide can be branched following an ubiquitination or be cyclic with or without branching. This type of modification can be the result of natural or synthetic post-translational processes that are well known to a person skilled in the art.

For example, a polypeptide modification may be, acetylation, acylation, ADP-ribosylation, amidation, covalent fixation of flavine, covalent fixation of heme, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bridge formation, demethylation, cysteine formation, pyroglutamate formation, formylation, gamma-carboxylation, glycosylation, GPI anchor formation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature: PROTEINS-STRUCTURE AND MOLECULAR PROPERTIES, 2^{nd} Ed., T. E. Creighton, New York, 1993; POST-TRANSLATIONAL COVALENT MODIFICATION OF PROTEINS, B. C. Johnson, Ed., Academic Press, New York, 1983; Seifter et al. “Analysis for protein modifications and nonprotein cofactors”, Meth. Enzymol. (1990) 182: 626-646; and Rattan et al. “Protein Synthesis: Post-translational Modifications and Aging”, Ann NY Acad Sci (1992) 663: 48-62.

“SNP” (Single Nucleotide Polymorphism) is defined as any natural variation of a base pair in a nucleotide sequence. Each SNP reflects the possibility of having two or more different bases in the same position in the nucleotide sequence of the candidate gene, resulting in the fact that at least two different alleles of the candidate gene may be found in the genome of individuals. Preferably, a SNP may be situated on a gene (coding and/or regulating nucleotide sequence).

In the sense of the invention, a SNP may be a change in the nature of a nucleotide, a deletion, an insertion or a repetition of one or more nucleotides in the nucleotide sequence.

A SNP, in a nucleotide sequence, can be coding, silent or non-coding. A coding SNP is a polymorphism in the coding sequence of a nucleotide sequence that involves a modification of at least one amino acid in the sequence of amino acids encoded by this nucleotide sequence. In this case, the term SNP applies equally, by extension, to a variation in an amino acid sequence. A silent SNP is a polymorphism included in the coding sequence of a nucleotide sequence that does not involve a modification of any amino acid in the amino acid sequence encoded by this nucleotide sequence. A non-coding SNP is a polymorphism included in the non-coding sequence of a nucleotide sequence. This polymorphism can notably be found in an intron, a splicing site, a promoter or an enhancer or a silencer sequence.

“Mutated nucleotide sequence” corresponds to the nucleotide sequence of a preselected candidate gene comprising a sequence variation such as a SNP. This mutated nucleotide sequence corresponds to a new allele of the gene revealed by the identification of a SNP in this sequence and that is preferably unknown in the prior art. By extension, a mutated protein corresponds to a protein encoded by said mutated nucleotide sequence.

“Functionality”: is the biological activity of a protein or a nucleotide sequence coding for said protein and/or the expression (level of expression) of a protein or a nucleotide sequence coding for said protein. The biological activity may, for example, be linked to the affinity or to the absence of affinity to a ligand or a receptor of a protein encoded by the nucleotide sequence of the preselected candidate gene. The functionality of the preselected candidate gene may be known or determined by a skilled person in the art.

“Functional SNP” is defined as a SNP, such as previously defined, which is included in the nucleotide sequence of a preselected candidate gene, and which modifies the functionality of the preselected candidate gene.

A functional SNP may increase, reduce or suppress the biological activity and/or the expression of the protein encoded by the nucleotide sequence of the preselected candidate gene or of this latter nucleotide sequence.

A functional SNP can equally induce a change in the nature of the biological activity of the polypeptide encoded by the nucleotide sequence of the preselected candidate gene or of this latter nucleotide sequence.

A functional SNP, for example located in the coding part of the nucleotide sequence that encodes for the signal peptide of the protein(s), may affect the activity at the proper localization and/or the localization of the protein(s) encoded by these genes.

A functional SNP may modify the expression of the candidate gene (at the level of transcription and/or translation) or of the protein(s) encoded by the gene (post-translational changes such as glycosylation for example).

A functional SNP may affect the expression and/or activity of the preselected candidate gene when it is positioned in a regulatory sequence of the gene such as, for example, in the promoter or enhancer.

A functional SNP is also any natural variation, situated in the coding sequence of a candidate gene and identified in the genome of one or more individuals of a random population, which causes either a stopping of translation (introduction of a STOP codon) or a change in the nature of an amino acid of the protein(s) encoded by this gene, if it or they exist, and which changes the activity of said protein(s). In this case, a variability in the activity (also called functionality) of the protein(s) encoded by the candidate gene in the random population is revealed.

“Common disease” is any disease in the general population, for which it is thought that more than one gene is involved in its appearance in patients and/or in a particular resistance to the development of this disease in certain individuals of the population. It is also called, for the same reasons, polygenic disease. This kind of human diseases may be, among others, the cancers; the cardiovascular diseases; any disease forming a risk factor for the cardiovascular diseases, such as, for example, diabetes type 1 and 2, hypertension, hypercholesterolemia or metabolic disease such as obesity; the autoimmune diseases; infectious diseases; diseases of the central nervous system such as, for example, Alzheimer's disease or schizophrenia or even depression; the rejection of tissue or organ graft(s); anemia; allergy or asthma.

The present invention concerns a method for determining at least one functional SNP in a gene, comprising the following steps:

a) Preselecting a candidate gene;

b) Providing a sample population comprising a significant number of individuals chosen substantially at random from the general population;

c) Isolating from each individual of the sample population at least one fragment of the nucleotide sequence of the preselected candidate gene;

d) Identifying at least one SNP in at least one fragment isolated in step c); and

e) From the SNP(s) identified in step d), identifying those with functionality.

Preferably, the significant number of individuals chosen substantially at random in the population in step b) is greater than 100, especially greater than 150, preferably greater than 200 and very particularly greater than 250. More preferably, the significant number of individuals chosen randomly in the population in step b) is comprised between 250 and 400.

The individuals may be selected by ethnic groups as will be seen hereafter in the methods section, and for each of these a significant number of individuals per ethnic group can be taken, thus forming the random population, for example greater than 5, especially greater than 10, preferably greater than 20 and very particularly greater than 100.

Preferably, the genotype and/or the phenotype of individuals chosen substantially at random in the population in step b) are not known, for example, by the experimenter.

In a preferred embodiment, individuals are chosen at random in the general population. It means that no specific characteristic are taken into account for the choice of individuals in order to provide the sample population. In this case, individuals are chosen at random without selecting any criteria.

A fragment of nucleotide sequence of the candidate gene is preferably isolated in step c) by a PCR or RT-PCR reaction. The Polymerase Chain Reaction (PCR) and the Reverse Transcriptase-Polymerase Chain Reaction (RT-PCR) are well known to the person skilled in the art.

The isolation of genomic DNAs can also be carried out by methods well known in the state of the technique.

Under preferential conditions of use of the above-described method, the fragments of specific DNAs corresponding to the predetermined fragments of regulatory and coding sequences of the candidate genes of individuals of the random population are amplified by chain polymerization reaction (PCR) by using appropriate oligonucleotide primers. Software such as Primer3® can be used to choose several pairs of primers making it possible to amplify the regions chosen by PCR (for example total or partial binding sequences of transcription factors in the promoters, total or partial splicing sequences of introns, total or partial sequences of exons).

The identification of a SNP in step d) may be carried out by at least one method selected from the group consisting of: direct sequencing, multiplexing method using denaturing high performance liquid chromatography (DHPLC), single strand conformation polymorphism (SSCP) (mentioned, for example, in Orita et al.; 1989; Genomics 5, 874-879), denaturing gradient gel electrophoresis (DGGE) (such as Myers et al.; 1987; Enzymol. 155, 501-527), methods based on the cleavage of the mismatch by chemicals or enzymes, allele-specific hybridization, allele-specific primer extension and allele-specific oligonucleotide ligation.

Under preferential conditions, the detection of the SNPs is carried out by DHPLC analysis. This methodology exploits the retention difference on a column of homo-duplex and hetero-duplex double-stranded species under conditions of partial thermal denaturation.

In fact, DHPLC generally detects SNPs with a greater effectiveness (97%) by comparison with sequencing (85 to 90%).

Such a procedure which involves the use of a multiplexing method of samples is described in FR-A-2,793,262 (Application No. 99 5651 of May 4, 1999).

Briefly, the amplified DNA fragments from the genomic DNA of heterozygous or homozygous individuals are separated under partially denaturing conditions by HPLC.

Preferably, the amplification products corresponding to several individuals may be mixed, preferably between 3 and 50 individuals, particularly between 3 and 5 individuals and very particularly 3 individuals, before proceeding with the denaturation and DHPLC analysis.

Other preferential conditions to use with the DHLPC and later steps of the procedure of the invention are described in FR-A-2,793,262.

The classification of the identical nucleotide sequences in homogeneous groups may be carried out by analysis of the profiles of the chromatograms obtained by DHPLC analysis. Identical nucleotide sequences are classified into homogeneous groups on the basis of similar DHPLC chromatograms.

Chromatography, especially DHPLC combined with sequencing makes it possible to locate each SNP on each nucleotide fragment and to characterize the nature of the bases associated with each polymorphism.

The identification of the polymorphism of the nucleotide sequence of heterozygous individuals in each group presenting a heterozygous chromatogram by comparison with the reference wild-type sequence is preferably carried out by sequencing the heterozygous nucleotide sequences. Sequencing is a procedure well known to the person skilled in the art and here it can be carried out, for example, by the technology of capillary sequencing well known to the person skilled in the art.

The identification of a SNP in step d) is preferably carried out by a multiplexing method using denaturing high performance liquid chromatography (DHPLC) followed by sequencing.

The determination of the functionality of the SNP(s) in step e) may be carried out by at least one method selected from among bioinformatic tools such as, for example, bioinformatic molecular modeling (in silico) and biological assay (in vivo or in vitro).

Preferably, the determination of the functionality of the SNP in step e) is carried out by comparison of functionality between:

i) a wild-type protein encoded by the reference wild-type nucleotide sequence of the preselected candidate gene, and

ii) a mutated protein encoded by the mutated nucleotide sequence of the preselected candidate gene comprising at least one SNP as identified in step d).

The determination of the functionality of a nucleotide sequence depends on the preselected candidate gene. Tools, such as bioinformatic tools, for example, enable a selection of the functional SNPs that are located in the regulatory sequences of the candidate genes which reveal a change in sequences known from the prior art as being important for the expression of the gene including, for example, the TATA and CAT boxes, sites known as enhancers, binding sites for transcriptional factors, and sites known as silencers.

A selection is also made of the functional SNPs that are located in the coding sequences of the candidate genes and that reveal the appearance of a STOP codon in these sequences and therefore an abnormal stop of the translation at the site of the functional SNPs.

Finally, a selection is made among all the identified SNPs between, on the one hand, the coding SNPs that induce a change in the nature of the amino acids of the protein(s) encoded by these genes and, on the other hand, the SNPs that do not cause a change in the nature of the amino acids of the proteins encoded by these genes.

The nature of the change in the sequence makes it possible to determine whether or not there is a coding of a different amino acid, and if it is different, one can examine whether this amino acid is essential to the function fulfilled by the corresponding protein.

In fact, the physicochemical nature of changes in the amino acids revealed by the coding SNPs can be determined, including the appearance or change in electric charge of the amino acid and the change of the hydrophilic or hydrophobic nature of the amino acid. The amino acids that are important for the activity of the protein and/or the domains, for which a relationship with a functional activity of the protein has been proven or is suspected, are identified.

Practically, that consists of listing all the proteins appearing in the same family in the human species or in the animal kingdom and therefore sharing the same functional activities (homologous, heterologous or orthologous) and often a comparable structure, at least at the level of one or more domains, then creating multiple alignments.

In addition, several databases are available in the public domain which list these functional domains in the form of units, patterns or matrices (PROSITE, BLOCKS, PFAM, etc.). Exhaustive research of the literature completes the group and particular attention is related to work relating to mutations observed or induced by self-directed mutagenesis and their involvement in the reported function of the protein. Functional SNPs found in the sequence of these important amino acids are particularly studied.

From methods known in the prior art, it is possible to determine the genomic organization of the gene to be studied, to localize the promoters, the exons and the introns as well as the sites known as “splicing” from the sequence of the candidate gene.

New functional SNPs are also selected among the coding SNPs when the change in the nature of the amino acid observed for a given coding SNP concerns an amino acid, or the signal peptide of the protein encoded by the candidate gene in the case where a signal peptide exists, making it possible to predict a change in the activity at the proper localization and/or a change in the localization of the corresponding protein, or when the coding SNP reveals the change in an amino acid which, in the prior art, is described as important for the structure of the corresponding protein(s).

By identifying the residues and/or domains preserved between species and/or between these proteins and/or domains, the mutations caused by the SNPs that are likely to affect the functional activity of the target can thus be predicted in silico.

The impact of the mutant allele revealed by this last type of SNP on the functional structure of the corresponding protein is then determined, for example, as a result of computer software allowing molecular modeling of both types of proteins, the reference wild-type and the mutant. Here each type of protein corresponds to one allele of the candidate gene.

Previous knowledge, according to the prior art, of the three-dimensional structure of the reference wild-type protein and of the amino acids involved in the activity of this protein enables the determination, with good reliability, of the change caused by the mutated allele comprising the functional SNP on the structure and therefore the function of the protein.

Also, the protein corresponding to the reference wild-type sequence and the mutated or mutant protein corresponding to the mutant allele can be produced by known methods.

By implementation of an appropriate test in vitro for example, biological or pharmacological, it can be deduced if the change caused by the mutated allele of the gene modifies or does not modify the function of the protein encoded by the candidate gene. Expression tests can also be developed in vitro (for example, expression tests of reporter genes such as the one coding for luciferase placed under the control of mutated regulatory sequences) to identify the mutant alleles comprising functional SNP(s) in the regulatory sequence of the candidate genes that modify the expression of said genes.

Combined with the annotations of the protein primary sequences the structural models of the targets can be constructed by using de-novo tools for modeling (for example: SEQFOLD/MSI), for homology (example: MODELER/MSI), minimization of the force fields (examples: DISCOVER, DELPHI/MSI) and/or molecular dynamics (example: CFF/MSI).

The three-dimensional structures of the variants can then be modeled and the consequences of these structural changes on the functional activity of the target predicted.

More particularly, the present invention concerns a method for determining at least one functional SNP in a gene, comprising the following steps:

a) Preselecting a candidate gene;

b) Providing a sample population comprising a significant number of individuals chosen substantially at random from the general population;

c) Isolating from each individual of the sample population at least one fragment of the nucleotide sequence of the preselected candidate gene;

d) Forming one or more mixtures comprising fragments isolated in step c) by randomly mixing fragments from one or more individuals;

e) Conducting an analysis for comparing, between them, the fragments of each mixture formed in step d) in order to determine whether said mixture has a heterozygous or homozygous profile;

f) Forming one or more homogeneous groups comprising at least one mixture analyzed in step e), each of said homogeneous group having an identical heterozygous or homozygous profile;

g) Identifying at least one SNP in:

i) at least one fragment from each homogeneous group having a heterozygous profile formed in step f),

ii) at least one fragment of at least one mixture having an heterozygous profile as determined in step e), and/or

iii) at least one fragment isolated in step c) from an individual incorporated in a mixture having an heterozygous profile as determined in step e);

h) From the SNP(s) identified in step g), identifying those with functionality.

Preferably, at least two mixtures are formed in step d).

In a preferred embodiment of the invention, mixtures formed in step d) comprise at least one individual, preferably between 3 and 50 individuals, particularly between 3 and 5 individuals and very particularly 3 individuals,

In step e), analysis to determine if a mixture has a homozygous or heterozygous profile, is carried out on the fragments of each mixture. If all the individuals of the mixture are homozygous (the two alleles are wild type for the preselected candidate gene), the mixture will have an homozygous profile. By contrast, for example, if at least one individual of the mixture is heterozygous (one wild type allele and one mutated allele for the preselected candidate gene), the mixture will have a heterozygous profile.

In step f), each mixture is classified as having a homozygous or heterozygous profile between them in order to form homogeneous groups.

The analysis conducted in step e) may be carried out by a multiplexing method using denaturing high performance liquid chromatography (DHPLC).

Preferably, the identification of a SNP in step g) is carried out by sequencing. As mentioned above, the sequencing may be carried out on at least one sample of individual or on at least one mixture of individuals.

According to the present invention, it also possible to identify functional polymorphisms by a method for determining of one or more functional polymorphisms in the nucleotide sequence of a preselected candidate gene in which:

a) the fragment of genomic nucleotide acids of the candidate gene is isolated from a significant number of individuals chosen randomly in the population,

b) a comparative analysis of the nucleotide acid sequences of the individuals studied is conducted,

c) the identical nucleotide acid sequences are classified into homogeneous groups, and

d) the polymorphism of the nucleotide acid sequence in each group is identified by comparison with the nucleotide sequence of the reference candidate gene.

The present invention concerns equally the genotyping of all or part of the nucleotide sequence of the preselected candidate gene comprising at least one SNP determined by the method as defined above, in at least one individual. The genotyping may be carried out by minisequencing.

A genotyping corresponds to the identification of the nature of the alleles present in the genome of an individual, it may reveal the presence of a SNP in an individual or a population of individuals.

The functional SNPs identified in the candidate genes in the random population may be genotyped in the same random population and a statistical analysis is then done of the frequency of each allele (allelic frequency) in the random population, which makes it possible to determine the importance of their impact in the various ethnic groups that form the random population.

The genotype data are analyzed to estimate the frequency of distributions of the different alleles observed in the populations studied. Even if the effort is related principally to the SNPs validated functionally, investigation of the linkage disequilibrium between the SNPs discovered in the random population may be carried out also with the nonfunctional SNPs that can nevertheless be associated with the more relevant functional SNPs, and therefore can be markers of the latter. These nonfunctional SNPs could be used for the development of diagnostic/prognostic kits as markers of the functional SNPs with which they will be in linkage equilibrium. The calculation of the allelic frequencies can be carried out with the aid of software such as SAS-suite® (SAS) or SPLUS® (Mathsoft). The comparison of the allelic distributions of the SNPs through different ethnic groups of the random population can be performed using the software ARLEQUIN® and SAS-suite®).

The present invention is also directed to a method for determination of the frequency of polymorphism of the nucleotide sequence identified above, in which the genotyping is carried out by minisequencing with ddNTPs hot (2 different ddNTPs labeled with fluorophores) and cold (2 unlabeled ddNTPs), in combination with a polarized fluorescence reader (FP-TDI Technology or Fluorescence Polarization Template-direct Dye-Terminator Incorporation) is well known to the person skilled in the art.

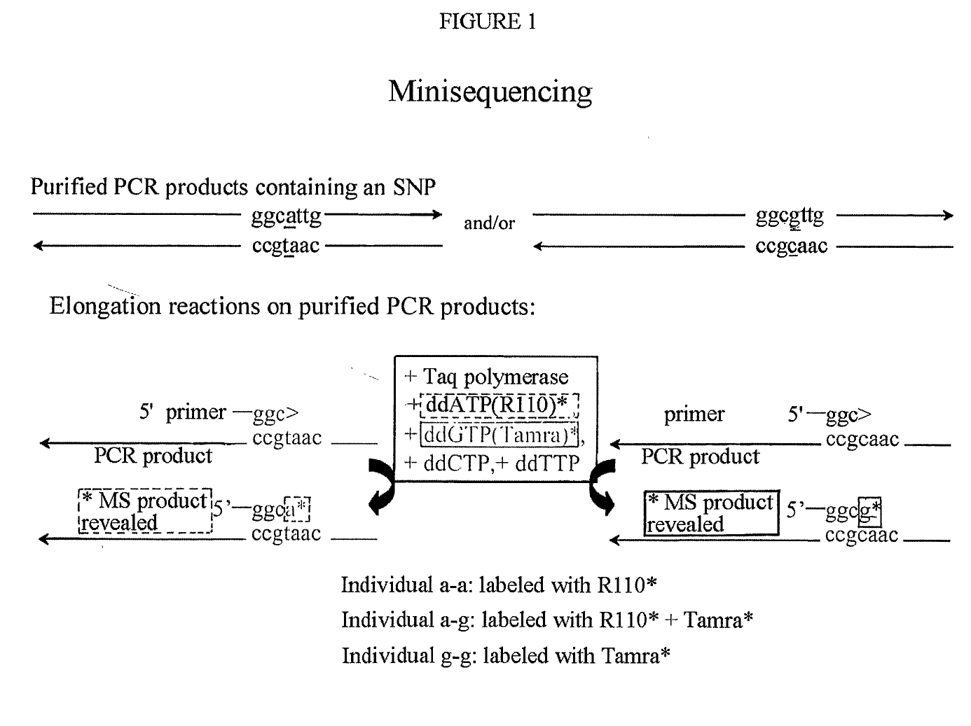

In this embodiment, genotyping is carried out on a product obtained after PCR amplification of the DNA of each individual, this PCR product being chosen to cover the gene region containing the SNP studied as is given in FIG. 1. After the last step of the PCR in the thermocycler, the plate is then placed on a polarized fluorescence reader for reading the labeled bases by using the specific excitation and emission filters of the fluorophores. The intensity values of the labeled bases are reported in a graph. Thus, up to four categories are obtained.

The present invention is also directed to a method for the genetic diagnosis of a disease or a resistance to a disease linked to the presence of a mutated nucleotide sequence of the preselected candidate gene in an individual comprising detecting the presence or absence in said individual of at least one functional SNP identified by the method of the invention.

The present invention is also directed to a method for the genetic diagnosis of a disease linked to the presence of one or several mutation(s) in the form of one or several mutant allele(s) comprising one or several functional SNP(s), to form a map of functional genetic markers taken in reference as well as showing a transgenic sequence (that is, different from the reference sequence) carried by said mutant allele in the nucleotide sequence of the candidate gene.

The present invention is also directed to a method for generating a map of genetic markers comprising performing the method for determining a functional SNP of the invention on at least one preselected candidate gene.

The present invention also makes it possible to form a map of functional genetic markers taken in reference for the development of pharmacogenetic or in other words, pharmacogenomic, tests for which genetic profiling of the individuals recruited for clinical trials will be carried out from the functional SNP markers taken in reference in order to identify the panel(s) of markers that will make it possible to differentiate the responding individuals, the non-responders or the individuals in whom the therapeutic molecules tested will have adverse effects, within the goal of optimizing said clinical trials for better effectiveness of the therapeutic molecules.

The present invention is also directed to a method for preparing a polynucleotide comprising the nucleotide sequence of the preselected candidate gene comprising at least one functional SNP, comprising the following steps:

a) Determining at least one functional SNP by the method of the invention; and

b) Producing a polynucleotide comprising a mutated nucleotide sequence of the preselected candidate gene comprising at least one functional SNP determined in step a).

The production of a polynucleotide mentioned in step b) may be carried out by standard DNA or RNA synthetic methods and/or by site-directed mutagenesis starting from the wild type nucleotide sequence of the preselected candidate gene by replacing the wild-type nucleotide by the mutated nucleotide.

Such a polynucleotide can equally include, for example, nucleotide sequences coding for pre-, pro- or pre-pro-protein amino acid sequences or marker amino acid sequences, such as hexa-histidine peptide.

This polynucleotide may equally be associated with nucleotide sequences coding for other proteins or protein fragments in order to obtain fusion proteins or other purification products. It can equally include nucleotide sequences such as the 5′ and/or 3′ non-coding sequences, such as, for example, transcribed or non-transcribed sequences, translated or non-translated sequences, splicing signal sequences, polyadenylated sequences, ribosome binding sequences or even sequences which stabilize mRNA.

The present invention is also directed to a method for preparing a polypeptide comprising an amino acid sequence of the preselected candidate gene comprising at least one coding functional SNP, comprising the following steps:

a) Determining at least one coding functional SNP by the method of the invention; and

b) Producing a polypeptide comprising a mutated amino acid sequence of the preselected candidate gene comprising at least one coding functional SNP determined in step a).

The production of a polypeptide mentioned in step b) may be carried out, for example, by standard methods of synthetic amino acid sequence production.

The present invention is also directed to a databank comprising functional SNPs determined by the method for determining of at least one functional SNP in the nucleotide sequence of a preselected candidate gene as defined above.

The present invention is also directed to a method for creating a databank of functional SNPs comprising performing the method for determining according to the invention, for at least one preselected candidate gene, and collecting said functional SNPs identified by said method.

The invention also concerns a method for identifying the functional SNP(s) associated with at least one pathology and/or disease or the resistance thereto, comprising analyzing the databank as defined above, for statistically relevant associations such as, for example, association with a genotype, a phenotype, a pathology or a disease and/or a resistance to a pathology or a disease.

Functional SNPs in the nucleotide sequences of the candidate gene may be used for the identification or determination of new potential diagnostic/prognostic or therapeutic targets in a random population for the prevention and treatment of common diseases.

The present invention is also directed to the use of a therapeutically effective amount of a polynucleotide and/or polypeptide prepared as defined above and a pharmaceutically acceptable carrier, for the preparation of a medicament, specifically for treating an individual having a pathology and/or disease correlated to the presence or absence of a mutated allele comprising at least one functional SNP in a gene linked to said pathology and/or disease.

The pharmaceutically acceptable carrier generally used in medicament or in pharmaceutical composition may be incorporated with a polynucleotide and/or a polypeptide prepared as defined above.

The present invention also concerns a method for treating an individual having a pathology and/or disease correlated to the presence or absence of a mutated allele comprising at least one functional SNP in a gene linked to said pathology and/or disease comprising administering a therapeutically effective amount of a polynucleotide and/or polypeptide prepared as defined above and a pharmaceutically acceptable carrier.

The present invention is also directed to a polynucleotide containing or corresponding to a mutated nucleotide sequence of a preselected candidate gene comprising at least one SNP revealed by the method of the invention.

Such a polynucleotide may be obtained from the reference wild-type sequence of the candidate gene by mutation of the base pair(s) of SNP(s) determined above by methods well known to the person skilled in the art and in particular by site-directed mutagenesis.

This polynucleotide may be incorporated into vectors. Different types of recombinant vectors can be used such as expression vectors in bacteria, mammalian cells or insect cells such as, for example, Drosophila cells.

These recombinant vectors can be used for transfecting cells so as to obtain transformed cells. Different types of cell lines can be used such as those described above. The introduction of nucleotide sequences determined above can be carried out by methods well known to the person skilled in the art and in the laboratory manuals such as Davis et al., Basic Methods in Molecular Biology (1986) and Sambrook et al., Molecular Cloning: A Laboratory Manual, 2^{nd} edition, Cold Spring Harbor Laboratory Press, Cold Spring Harbor, N.Y. (1989). The host cells can be bacteria, fungi, yeasts, insect cells, plant cells or animal cells such as CHO, COS, HeLa, C127, 3T3, BHK and HEK 293.

The present invention is also directed to a protein corresponding to a polypeptide comprising a mutated amino acid sequence of a preselected candidate gene comprising at least one SNP revealed by the method of the invention.

The proteins identified by the present method can be used in methods to determine new compounds with a positive (activating) or negative (inhibiting) effect on the activity of said protein. Such methods involve the use of host cells described above in the presence of candidate compounds for experimentation. The determination of the effect produced by these candidate compounds can be carried out by experimentation such as, for example, a binding test between the candidate compound and the host cell, or a test demonstrating the activation or inhibition effect of the candidate compound on a signal caused by said protein in the host cell.

The identification of the functional SNPs thus enables post-genomic or post-sequencing research of the human genome for the identification of new therapeutic targets which will make it possible to develop diagnostic or prognostic kits for the associated diseases, such as new therapeutic molecules.

The present invention also makes it possible to develop therapeutic molecules such as antibodies, vectors of gene therapy and active molecules determined from the structure of the mutated protein(s) encoded by the mutated allele(s) comprising one or more functional SNPs connected with the appearance of or resistance to common diseases in the population, for treatment of these same diseases.

The present invention is also directed to an active molecule characterized in that it is developed from a protein as described above for the prevention or the treatment of diseases and pathologies.

The present invention further concerns a medicament containing a protein defined previously as active ingredient and a pharmaceutically acceptable carrier.

The present invention is also directed to a method for identifying the cause of or resistance to a pathology and/or disease comprising determining at least one functional SNP by the method of the invention and studying the involvement of said functional SNP(s) in said pathology and/or disease.

The present invention is also directed to a method for determining whether an individual is predisposed or resistant to a pathology and/or disease comprising determining at least one functional SNP by the method of the invention and identifying if the genome of said individual has a mutated allele comprising said functional SNP(s).

Methods

### EXAMPLE 1

### Determination of Functional SNPs in the Nucleic Sequence of the Gene Encoding for Human Interferon Alpha 2 (IFNα-2)

Stage a): Preselection of the Reference Sequence of the Candidate Gene

The sequence and genomic organization of the gene encoding for human interferon alpha-2 have been deposited since 1994 under the name of “interferon alpha-a” in the GenBank bank of NCBI under the code “J00207.” This sequence is used as “reference wild-type sequence” and the numbering of the positions on nucleotides mentioned below are related to this sequence. The coding region (CDS) of this gene comprises 567 base pairs (bp) and encodes for a protein with 189 amino acids.

The alpha interferons compose an excessively close family in terms of protein sequences as much in man as in all higher mammals. This is demonstrated when the sequences of these proteins are aligned by a tool such as ClustaIW.

Stage b): Isolation of the Genomic DNA of the Preselected Candidate Gene in a Random Population of Individuals

To discover the SNPs according to the detailed method of the invention, a population of individuals taken substantially at random has been screened (not selected on a particular phenotypic criterion such as collection of medical, clinical, epidemiological, physiological, age, sex or biological data) and called random population.

The genomic DNAs of the individuals of the tested population have been provided by the Coriell Institute in the United States.

The individuals are distributed as follows:

| PHYLOGENIC | SPECIFIC ETHNIC | NUMBER OF |
|---|---|---|
| POPULATION | POPULATION | INDIVIDUALS |
| African American | African American | 50 |
| Amerind | Southwestern | 5 |
| | American Indian | |
| | South American (Andes) | 10 |
| Caribbean | Caribbean | 10 |
| European Caucasoid | North American Caucasian | 79 |
| | Iberian | 10 |
| | Italian | 10 |
| Mexican | Mexican | 10 |
| Northeast Asian | Chinese | 10 |
| | Japanese | 10 |
| Non-European Caucasoid | Greek | 8 |
| | Indo-Pakistani | 9 |
| | Middle-Eastern | 20 |
| Southeast Asian | Pacific Islander | 7 |
| | South Asian | 10 |
| South American | South American | 10 |

The primers used to clone, by polymerase chain reaction (PCR), the gene encoding for the human interferon alpha-2 are the following:

| | | Melting | | | |
|---|---|---|---|---|---|
| Gene Fragment | Primers | Temperature | Start/Stop | Length | Sequence |
| F1 | Forward primer | 55.25 | 3 | 20 | GCCTCTTATGTACCCACAAA |
| | | | | | [SEQ ID NO. 1] |
| F1 | Reverse primer | 56.43 | 537 | 20 | CACCAGTAAAGCAAAGGTCA |
| | | | | | [SEQ ID NO. 2] |
| F2 | Forward primer | 56.03 | 4700 | 20 | CACCCATTTCAACCAGTCTA |
| | | | | | [SEQ ID NO. 3] |
| F2 | Reverse primer | 55.77 | 1124 | 19 | AGCTGGCATACGAATCAAT |
| | | | | | [SEQ ID NO. 4] |

Start/stop: beginning (sense) or stop (antisense) of the primers by comparison with the reference sequence.

The specificity of these two couples of primers has been tested and it appeared that no other fragment than that expected was found. These primers have made it possible to amplify two fragments, named F1 and F2, of, respectively 535 bp and 655 bp in length, for which the sequence is given below. F2 covers the coding sequences of the IFNα-2 gene, which is indicated by undelining in the sequence of this fragment.

Sequence of F1: [SEQ ID NO.5]
gcctcttatgtacccacaaaaatctattttcaaaaaagttgctctaagaatatagttatcaagttaagtaaaatgtc
aatagccttttaatttaatttttaattgttttatcattctttgcaataataaaacattaacutatactttttaatttaatgtata
gaatagagatatacataggatatgtaaatagatacacagtgtatatgtgattaaaatataatgggagattcaatc
agaaaaaagtttctaaaaaggctctggggtaaaagaggaaggaaacaataatgaaaaaaatgtggtgaga
aaaacagctgaaaacccatgtaaagagtgtataaagaaagcaaaaagagaagtagaaagtaacacagg
ggcatttggaaaatgtaaacgagtatgttccctatttaaggctaggcacaaagcaaggtcttcagagaacctgg
agcctaaggtttaggctcacccatttcaaccagtctagcagcatctgcaacatctacaatggccttgacctttgctt
tactggtg
Sequence of F2 [SEQ ID NO.6]
cacccatttcaaccagtctagcagcatctgcaacatctacaatggccttgacctttgctttactggtggccctcctg
gtgctcagctgcaagtcaagctgctctgtclggctgtgatctgcctcaaacccacagcctgggtagcaggagga
ccttgatgctcctggcacagatgaclgagaatctctcttttctcctgcttgaaggacagacatgactttggatttccc
caggaggagtttggcaaccagttccaaaaggctgaaaccatccctgtcctccatgagatgatccagcagatct
tcaatctcttcagcacaaaclqactcatctgctgcttgggatgagaccctcctagacaaattctacactgaactct
accagcagctgaatgacctggaagcctgtgtgatacagggggtgggggtgacagagactcccctgatgaag
gaggactccattctggctgtgaggaaatacttccaaagaatcactctctatctgaaagagaagaaatacagcc
cttgtgcctgggaggttgtcagagcagaaatcatgagatctttttgtttgtcaacaaacttgcaagaaagtttaag
aagtaaggaatgaaaactggttcaacatggaaatgattttcattgattcgtatgccagct

The results obtained during analysis of the coding fragment F2 are presented.

Materials

Autoclaved water

10×PCR buffer (delivered with the enzyme) Gibco

MgSO₄ 50 mM

Platinum Taq enzyme 5 U/μL

dNTP 100 mM

Forward and Reverse primers

Genomic DNA 1 ng/μL

Plate 96 wells (Costar)

Plate 384 wells (ABGene)

PCR reaction: ×plates 96 wells or 384 wells per fragment to be amplified according to the number of individuals to be tested.

| | | | | | Vol. |
|---|---|---|---|---|---|
| | | | | | per |
| | | | Used | Final | well |
| Product | Supplier | Reference | concentration | concentration | (μl) |
| Buffer | Gibco | 11304-029 | 10× | 1× | 2.5 |
| MgSO₄ | Gibco | 11304-029 | 50 mM | 0.02 M | 1.075 |
| dNTP | Gibco | 10297-018 | 10 mM | 0.2 mM | 0.5 |
| Primer F | Gibco | | 10 μM | 0.2 μM | 0.5 |
| Primer R | Gibco | | 10 μM | 0.2 μM | 0.5 |
| H₂O | | | | | 14.85 |
| Enzyme | Gibco | 11304-029 | 5 U/μl | 0.375 U | 0.075 |
| DNA | | | 1 ng/μl | | 5 |
| Final | | | | | 25 |
| volume | | | | | |

Programming the thermocylers (Tetrad MJ research):

| | | |
|---|---|---|
| 1 cycle: | 94° C. | 1 min |
| 35 cycles: | 94° C. | 15 sec |
| | 56° C. | 30 sec |
| | 68° C. | 1 min |

After testing the PCR products on 2% agarose gel, the amplified products are denatured on Thermocyclers (Tetrad from MJ Research) according to the cycle program:

| | | |
|---|---|---|
| 1 cycle: | 95° C. | 3 min |
| 1 cycle: | 95° C. | 1 min |

This is followed by a series of cycles by decreasing the temperature 1.6° C./cycle to 25° C.).

Once denatured, the samples are multiplexed by three on 96-well plate.

Stage c): Study of the DNA Sequence of Each Individual

The PCR products were analyzed by DHPLC (denaturing high performance liquid chromatography).

Buffer A: for 1 liter

250 μL acetonitrile (ACN)

50 mL triethylammonium (TEAA) 2 M

Buffer B: for 1 liter

250 mL acetonitrile (ACN)

50 mL triethylammonium (TEAA) 2 M

The column is equilibrated under the following buffer conditions:

50% buffer A

50% buffer B

with a program flow of 0.9 mL/min.

The performances of the column are tested:

on the one hand, at 50° C. by injection of 5 μL pUC 18 digested by the restriction enzyme Hae III with a buffer flow of 0.75 mL/μL and a gradient of 43% buffer B and 57% buffer A,

on the other hand, at 56° C. by injection of 5 μL of a mutation standard with a buffer flow of 0.9 mL/μL and a gradient of 47% buffer B and 53% buffer A.

The study of sequences by the software Wave Maker® (Transgénomique Inc.) gave information on the temperature and the buffer gradient according to which the samples must be treated. Trial tests were carried out in order to establish the effective conditions for analysis of the sequences.

Therefore, with the temperature(s) and gradient conditions of buffer A and B, 3 μL of each of the 96 samples are analyzed over 14 h in the DHPLC machines called Waves® (Transgénomique Inc.)

The analysis of the fragments requires specific temperatures accompanied by buffer gradients listed in the table below, obtained by the software Wave Maker®) (Transgénomique Inc.).

| | % A | % B | % C | Flow |
|---|---|---|---|---|
| Time (min) | (0.025% ACN) | (25% ACN) | (75% ACN) | (ml/min) |
| 0 | 45 | 55 | 0 | 0.9 |
| 0.1 | 40 | 60 | 0 | 0.9 |
| 4.1 | 32 | 68 | 0 | 0.9 |
| 4.2 | 0 | 100 | 0 | 0.9 |
| 4.7 | 0 | 100 | 0 | 0.9 |
| 4.8 | 45 | 55 | 0 | 0.9 |
| 6.8 | 45 | 55 | 0 | 0.9 |

The equilibrated column is tested with proposed conditions by the Wave Maker® (Transgénomique Inc.). These conditions are made effective during the final analysis of the F2 fragment of the samples.

The chromatograms obtained are then analyzed.

The analysis of the chromatographic profiles obtained made it possible to detect the heterozygous and the homozygous among the individuals of the tested population on the basis of the chromatograms or even “profiles” of different forms. Certain profiles have made it possible to establish families (groups) of individuals presenting similar chromatograms.

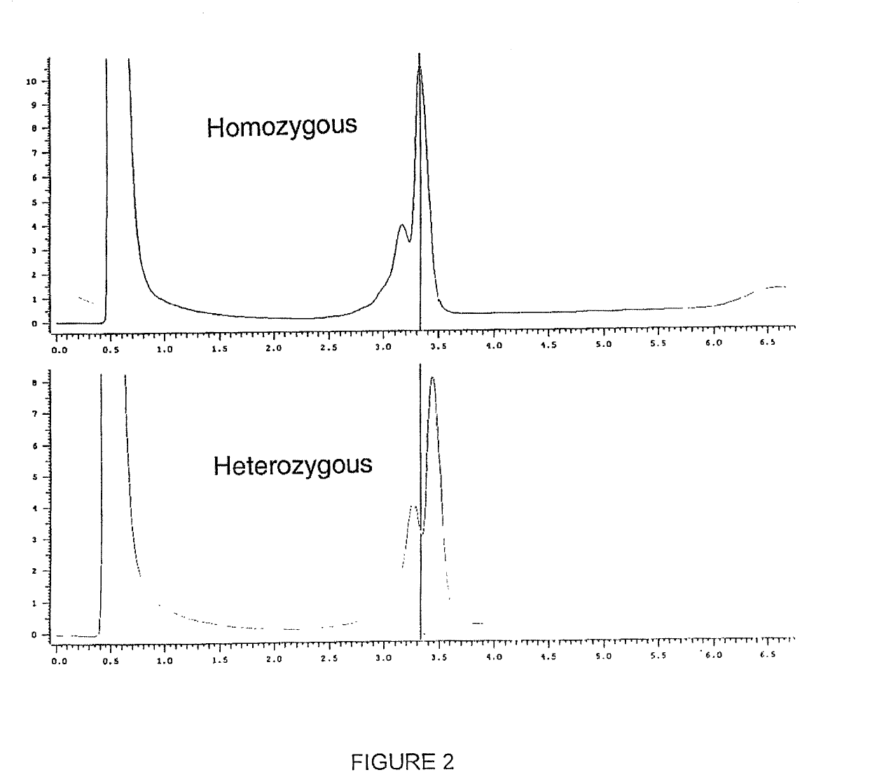

A wild-type profile corresponding to a homozygous individual in FIG. 2 (top part)

A different profile corresponding to a heterozygous individual (chromatogram in FIG. 2 (bottom part)).

Stage d): Sequencing of the DNA from Each Group

Next, one proceeds with sequencing the PCR products, by capillary on the ABI-PRISM 3700 DNA sequencers, corresponding to the heterozygous profiles.

Sequencing profile on the basis of a 96-well plate

Purification of the PCR products:

Weigh 50 g of Biogel P100 Fine. Suspend in 1 liter of ultrapure water. Leave standing for 8 h. Shake. Fill multiscreen “filtering bottom” plate (Biogel P100 Fine): 400 mL per well. Superimpose on recovery plate. Centrifuge: 500 g, 3 min. Replace the recovery plate with 1 new Greiner plate, superimpose with the aid of a Millipore adaptor. The PCR products are deposited on the P100. Centrifuge at 500 g, 4 min. Store at −20° C.

Sequencing reaction:

Sequencing consists of a new PCR reaction. A sequencing reaction corresponds to the following proportions: per well containing the multiplex of fragments amplified for the detection of SNP by DHPLC from three different individuals.

1 μL Big Dye Terminator

1 μL 5×buffer (tris-HCl 400 mM//MgCl2 10 mM)

10 ng PCR products for 100 bp (base pairs)

6 pmol primer

H₂O qsp 10 μL

It is centrifuged briefly.

Reaction cycles:

Denaturation 95° C./5 min

95° C./10 sec

Tm/5 sec

60° C./4 min

25 cycles. Duration: 2.5 h

Purification of the sequencing products:

Weigh 50 g of Sephadex G50 Super-Fine. Suspend in 1 liter of ultrapure water. Leave standing 8 h. Shake. Fill multiscreen “filtering bottom” plate (Biogel P100 Fine): 400 mL per well. Superimpose on a recovery plate. Centrifuge: 1500 g, 3 min. Replace the recovery plate with a new special “Optical” plate, DNA capillary sequencing machine ABI-PRISM 3700. 10 μL ultra-pure water per well are added to the plate leaving the sequencing reaction. Pour the so-diluted sequencing products on the G50. Centrifuge at 1200 g, 3 min. Store at −20° C.

Migration of the samples:

Migration is done on the capillary sequencer ABI-PRISM 3700 DNA.

Analysis is performed using the following methods: The “Optical” plate containing the samples is recovered and it is covered with an adhesive aluminum foil. Place the plate on an adapted rack in the ABI-PRISM 3700 DNA capillary sequencer and put it all in a free carrier A, B, C or D. Verify the levels of the buffer, water, polymer, isopropanol. Adjust them if necessary.

In the START menu, PE Biosystems tab, under subfile “3700 Programs”, open “Data Collection.” In the “Plate set up” tab, import the operation sheet by clicking on “import.” Assign the operation sheet by clicking on the carrier containing a large question mark, which carrier corresponds to the sequencing plate. When it is active, click on the green arrow. Time of trial: 4 h.

Control of the sequences:

In the START menu, PE Biosystems tab, open “Data Extraxtor.” Click on “Extract Now.” In the START menu, BE Biosystems tab, open “Sequencing Analysis 3.6.” Click on <<add files>> and import the previously extracted sequences. Open the sequences one by one and verify the quality of the electrophorograms, that is, the quality of migration of the sequences in the capillaries, the length of reading, and estimate the percentage of readable sequences. Transfer the sequences into the computer network, file “Sequencing—Sequences Discovery,” for identification of the SNPs.

With the aid of the sequences and with the “PolyPhred” software for analysis of the sequences the nucleotide nature and the position of the polymorphism have been identified. Eleven SNPs have been identified by this method. For example, in position 680 of the reference wild-type sequence of the gene encoding for interferon alpha 2, base A is replaced by G in a pool of 3 individuals in a random population. The overlay of the peaks is informative of the SNP.

Stage e): Determination of the Functional SNPs

Functional annotation was performed to precisely position the SNPs on the gene sequence and predict the effect of the SNPs on the activity of the gene. Among the eleven SNPs identified previously, six were on the promotor region, and five were in the coding region, from which three caused an amino acid change in the sequence of the protein encoded by IFNα-2 gene. This first step allows us to pre-select the SNPs for which a functional study will be carried out to determine if they are functional.

Here is examplified the functional study on two of the SNPs that cause an amino acid change in the protein sequence:

a680g corresponding to the amino acid change H57R on the immature protein encoded by the IFNα-2 gene (also further called H34R if one refers to the position of the amino acid on the mature protein), and

g1023a corresponding to the amino acid change M171 I on the immature protein encoded by the IFNα-2 gene (also further called M148 I if one refers to the mature protein sequence).

### EXAMPLE 2

### Determination of the Functionality of H34R (a680g)

a) Bioinformatic Modeling

The H34 residue is highly conserved for all the IFNs alpha sequences, except for the IFN alpha16 sequence for which a tyrosine is found at this position. This high conservation suggests an important role of H34 residue in the function of the protein. The H34 residue is described by J Piehler et al. (Journal of Biological Chemistry; JBC, September 2000) as participating in the binding domain of this interferon to its receptor (receptor-2 of the interferons). The work of J Piehler consisted of doing systematic self-directed mutagenesis by replacing several residues of this region with alanines. In the case of the H34A mutation J Piehler observes a significant decrease in the ability of this interferon to interact with its receptor. The structure of monomeric interferon α 2 determined by NMR is known and available in the PDB database (http://www.rcsb.org.pdb/) under the code 1ITF.

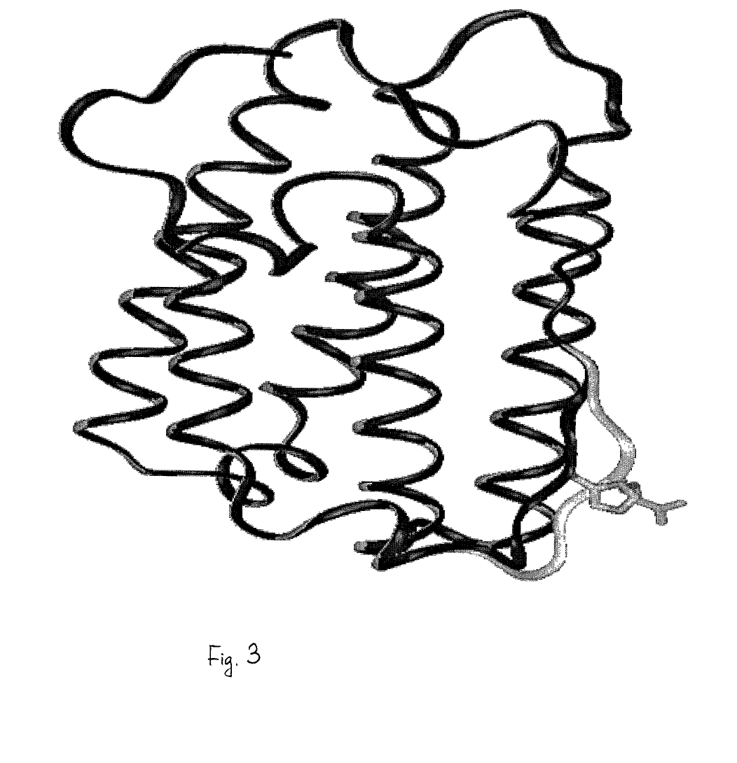

MODELER (MSI) was used to replace the histidine by an arginine at position 34 of the mature protein sequence. Such a modeling is represented in FIG. 3.

The residue at position 34 is located in the AB loop, accessible to the solvent and very near, in the spatial conformation, to the arginine of position 33, which is itself involved in the binding to the receptor according to Pieler's work. Replacing the histidine of position 34 by an arginine modifies the hydrogen bonds with the aspartate of position 32 and the tyrosine of position 129. Thus, the H34R mutation causes a weak modification of the AB loop but a strong modification of the hydrogen bonds network and the formation of several salt bridges: R33-E146, R34-E132, D35-R125. It is very likely that this SNP may cause strong functional disturbances.

b) Genotyping of the H57R functional SNP

The technique used for genotyping is fluorescent minisequencing, FP-TDI technology or Fluorescence Polarization Template-direct Dye-terminator Inc. Principle of minisequencing: Genotyping of the SNPs is based on the principle of minisequencing in which the product is detected by reading polarized fluorescence. Minisequencing consists of elongating an oligonucleotide, placed just upstream of the polymorphic site, by fluorolabeled dideoxynucleotides with the aid of a polymerase enzyme as illustrated in FIG. 1. The result of this elongation is analyzed directly by polarized fluorescence reading.

Steps of the protocol:

Minisequencing is carried out on a product obtained after PCR amplification of a sequence fragment of the IFNα2 gene which carries the functional SNP from the genomic DNA from each individual of the random population. This PCR product is chosen to cover the gene region containing the SNP studied. Then the PCR primers and the unincorporated dNTPs are eliminated before carrying out the minisequencing. All these steps, as well as the reading, are carried out in the same plate.

Genotyping requires 5 steps:

1) Amplification by PCR

2) Purification of the PCR product by enzymatic digestion

3) Elongation of the oligonucleotide

4) Reading

5) Interpretation of the reading

1) The PCR amplification of the sequence of the IFNα2 gene which covers the gene region containing the functional SNP is done with the aid of the same primers as those used for the identification of the SNPs. Therefore, the PCR product is made for each individual of the random population as described above in the step for the discovery of the functional SNP. This PCR product acts as matrix for the minisequencing reactions which are used to genotype the individuals for the functional SNP. The PCR product is carried out in the same plate. The reaction volume is 5 μL as described in the following table:

| | | | | Volume | |
|---|---|---|---|---|---|
| | | | | per | |
| | | | Initial | tube | Final |
| Supplier | Reference | Reagent | concentration | (μl) | Concentration |
| Life | Delivered with | Buffer (X) | 10 | 0.5 | 1 |
| Technologie | Taq | | | | |
| Life | Delivered | MgSO₄ (mM) | 50 | 0.2 | 2 |
| Technologie | with Taq | | | | |
| AP Biotech | 27-2035-03 | dNTP (mM) | 10 | 0.1 | 0.2 |
| Life | On request | Forward | 10 | 0.1 | 0.2 |
| Technologie | | primer (μM) | | | |
| Life | On request | reverse | 10 | 0.1 | 0.2 |
| Technologie | | primer (μM) | | | |
| Life | 11304-029 | platinium | 5 U/μl | 0.02 | 0.1 U/reaction |
| Technologie | | Taq | | | |
| | | H₂O | Qsp 5 μl | 1.98 | |
| | | DNA | 2.5 ng/μl | 2 | 5 ng/ |
| | | | | | reaction |
| | | Final | | 5 μl | |
| | | Volume | | | |

These reagents are distributed in a black PCR plate with 384 wells provided by ABGene (ref.: TF-0384-k). Once filled, the plate is sealed, centrifuged then placed in a thermocycler for 384 plate (Tetrad from MJ Research) and subjected to the following incubation: PCR cycles: 1 min at 94° C., followed by 36 cycles composed of 3 steps (15 sec at 94° C., 30 sec at 56° C., 1 min at 68° C.).

2) The PCR is then purified with the aid of two enzymes, shrimp alkaline phosphatase (or Shrimp Alkaline Phosphatase SAP) and exonuclease I (Exo I). The first of these enzymes enables the dephosphorylation of the dNTPs not incorporated during the PCR, while the second eliminates the single-stranded residues of DNA and therefore the primers not used during the PCR. This digestion is done by addition to the PCR plate of 5 μL reaction mixture that is prepared as described in the following table:

| | | | | Vol. | |
|---|---|---|---|---|---|
| | | | | per | |
| | | | Initial | tube | Final |
| Supplier | Reference | Reagent | Concentration | (μl) | Concentration |
| AP | E70092X | SAP | 1 U/μl | 0.5 | 0.5/ |
| Biotech | | | | | reaction |
| AP | 070073Z | Exo I | 10 U/μl | 0.1 | 1/ |
| Biotech | | | | | reaction |
| AP | Delivered | Buffer | 10 | 0.5 | 1 |
| Biotech | with SAP | SAP (X) | | | |
| | | H₂O | Qsp 5 μl | 3.9 | |
| | | PCR | | 5 μl | |
| | | Final | | 10 μl | |
| | | Volume | | | |

Once filled, the plate is sealed, centrifuged then placed in a thermocycler for 384 plate (Tetrad from MJ Research) and subjected to the following incubation: SAP-EXO digestion: 45 min at 37° C., 15 min at 80° C.

3) The elongation or minisequencing step is then carried out on this digested PCR product by the addition of a reaction mixture prepared as given in the table below:

| | | | | Vol. | |
|---|---|---|---|---|---|
| | | | | per | |
| | | | Initial | tube | Final |
| Supplier | Reference | Reagent | Concentration | (μl) | Concentration |
| Own | | Elongation buffer* | 5 | 1 | 1 |
| preparation | | (X) | | | |
| Life | On request | primer Miniseq (μM) | 10 | 0.5 | 1 |
| Technologies | | | | | |
| AP Biotech | 27-2051 | **ddNTPs (μM) | 2.5 of each | 0.25 | 0.125 |
| | (61,71,81)- | (2 cold ddNTPs) | | | of each |
| | 01 | | | | |
| NEN | Nel 472/5 | **ddNTPs (μM) | 2.5 of each | 0.25 | 0.125 |
| | and Nel | (2 labeled ddNTPs | | | of each |
| | 492/5 | (Tamra and R110) | | | |
| AP Biotech | E79000Z | Thermo-sequenase | 3.2 U/μl | 0.125 | 0.4 U/ |
| | | | | | reaction |
| | | H2O | Qsp 5 μl | 3.125 | |
| | | Digested PCR | | 10 μl | |
| | | Final volume | | 15 μl | |

The 5×elongation buffer is composed of 250 mM Tris-HCl pH 250 mM KCl, 25 mM NaCl, 10 mM MgCl2 and 40% glycerol

For the ddNTPs, a mixture of 4 bases is carried out according to the polymorphism studied. Only the 2 bases of interest (A/G) composing the functional SNP bearing a labeling either with Tamra or R110 ex SNP A/G; the mixture of ddNTP is composed of:

2.5 μM cold ddCTP,

2.5 μM cold ddTTP,

2.5 μM ddATP (1.825 μM ddATP and 0.625 μM ddATP labeled with Tamra),

2.5 μM ddGTP (1.825 μM ddATP and 0.625 μM ddATP labeled with R110).

Once filled, the plate is sealed, centrifuged, then placed in a thermocycler for 384 plates (Tetrad from MJ Research) and subjected to the following incubation: Elongation cycles: 1 min at 93° C., followed by 35 cycles composed of 2 steps (10 sec at 93° C., 30 sec at 55° C.).

After the last step in the thermocycler the plates is placed directly on an Analyst® HT polarized fluorescence reader from LJL Biosystems Inc. The plate is read with the aid of Criterion Host® software by using two methods. The first makes it possible to read the base labeled with Tamra by using specific excitation and emission filters of this fluorophore (excitation 550-10 nm, emission 580-10 nm) and the second makes it possible to read the based labeled with R110 by using the specific excitation and emission filters of this fluorophore (excitation 490-10 nm, emission 520-10 nm). In both cases, a dichroic double mirror (R110/Tamra) is used and the other reading parameters are:

Z-height: 1.5 mm

Attenuator: out

Temps d'intégration: 100,000 μsec

Raw data units: counts/sec

Switch polarization: by well

Plate settling time: 0 msec

PMT setup: Smart Read (+), sensitivity 2

Dynamic polarizer: emission

Static polarizer: S

A result file is then obtained containing the calculated values of mP for the Tamra filter and that for the R110 filter. These mP values are calculated from values of intensity obtained on the parallel plane (//) and on the perpendicular plane (⊥) according to the following formula:

mP=1000(//−g.⊥)/(//+g.⊥).

In this calculation the value on the filter ⊥ is weighted with a factor g. This is a parameter that must be previously determined experimentally.

4) and 5) Interpretation of the reading and determination of the genotypes

The mP values are reported on a graph with the aid of the Excel software from Microsoft Inc., or maintaining with the Allele Caller® software developed by LJL Biosystems Inc. On the abscissa is given the mP value of the base labeled with Tamra, on the ordinate is given the mP value of the base labeled with R110. A high mP value indicates that the base labeled with this fluorophore is incorporated and, conversely, a low mP value reveals the absence of incorporation of this base. Up to four categories are obtained. Once the locating of the different groups is made, the use of the Allele Caller® software, makes it possible to directly extract the defined genotype for each individual in the form of a table.

The sequences of both minisequencing primers necessary for the genotyping have been determined. These primers are selected to correspond to the 20 nucleotides placed just upstream of the SNP polymorphic site. Because the PCR product containing SNP is a product of double-stranded DNA, the genotyping can therefore be done either on the sense strand or the antisense strand. The primers selected are produced by Life Technologies Inc. The minisequencing primer of the SNP A211G of the fragment F2 was first validated on 16 samples then genotyped on the entire random population composed of 239 individuals and 10 negative controls.

The minisequencing primers are the following:

Sense primer: ctcctgcttgaaggacagac [SEQ ID NO. 7]

Antisense primer: cctggggaaatccaaagtca [SEQ ID NO. 8]

The following condition has been tested for minisequencing and retained for genotyping: Sense primer+ddTTP-R110+ddCTP-Tamra

Results:

Genotyping of the individuals from the random population was carried out by using the condition described previously. The genomic DNA of individuals of the random population (see stage b) of Example 1) were provided by the Coriell Institute of the United States.

After complete execution of the genotyping process, the determination of the genotypes of the individuals of the random population studied here for the functional SNP was carried out. This genotype is in theory either homozygous AA, or heterozygous AG, or homozygous GG in the individuals tested. In reality and as shown below, the homozygous GG genotype is not detected in the random population.

All of the 7 negative controls which have been tested have been validated. Of 239 individuals who have been tested, a genotype could be given for 236 individuals. Thus, the percentage of success of the genotyping reaches 99.2%.

The distribution of the genotypes determined in the random population and the calculation of the different allelic frequencies for this functional SNP are presented in the following table:

Distribution of genotypes

| Number of TT | Number of TC | Number of CC |
|---|---|---|
| 232 | 4 | 0 |

| | |
|---|---|
| Genotype | Allele |
| Frequency (%) | frequency (%) |

| | | | | |
|---|---|---|---|---|
| TT | TC | CC | T | C |
| 98.3 | 1.7 | 0 | 99.2 | 0.8 |

The definition of “allele frequency” or “genotype frequency” is the estimated frequency of a given allele or genotype in a population.

It is necessary to specify that allele T read as antisense corresponds to allele A read as sense or to the presence of histidine in position 57 of the IFNα-2 and therefore that the allele C read as antisense corresponds to the allele G read as sense corresponding to an arginine for this position in the corresponding sequence of the protein.

By examining these results by population it is noted that the 4 heterozygous individuals are all derived from a single subpopulation or ethnic group, the “African American” subpopulation of the random population. The analysis of this functional SNP in this population is the following:

| Distribution of genotypes | | | | | | Allelic | |
|---|---|---|---|---|---|---|---|
| Number | Number | Number | Genotype frequency (%) | | | frequency (%) | |
| of TT | of TC | of CC | TT | TC | CC | T | C |
| 45 | 4 | 0 | 91.8 | 82 | 0 | 95.9 | 4.1 |

### EXAMPLE 3

### Determination of the Functionality of M148 I (g1023a)

The M148 residue is highly conserved for all the IFNs alpha sequences, suggesting an important role of M148 residue in the function of the protein. The M148 residue is described by J Piehler et al. (Journal of Biological Chemistry; JBC, September 2000) as participating in the binding domain of this interferon to its receptor (receptor-2 of the interferons).

a) Modeling of a protein encoded by the mutated nucleotide sequence and the protein encoded by the nucleotide sequence of the reference wild-type gene

In a first step the three-dimensional structure of IFNα-2 has been constructed from that of human IFNα-2 for which the structure is available in the PDB database (code 1ITF) by using the software Modeler (MSI, San Diego, Calif.).

The mature polypeptide fragment was then modified so as to reproduce the observed mutation.

About a thousand steps of molecular minimizations were conducted on this structure by using the programs AMBER and DISCOVER (MSI).

Two series of calculations of molecular dynamics were then carried out with the same program and the same force fields.

In each case, 50,000 steps have been calculated at 300 K, terminated by 300 equilibration steps.

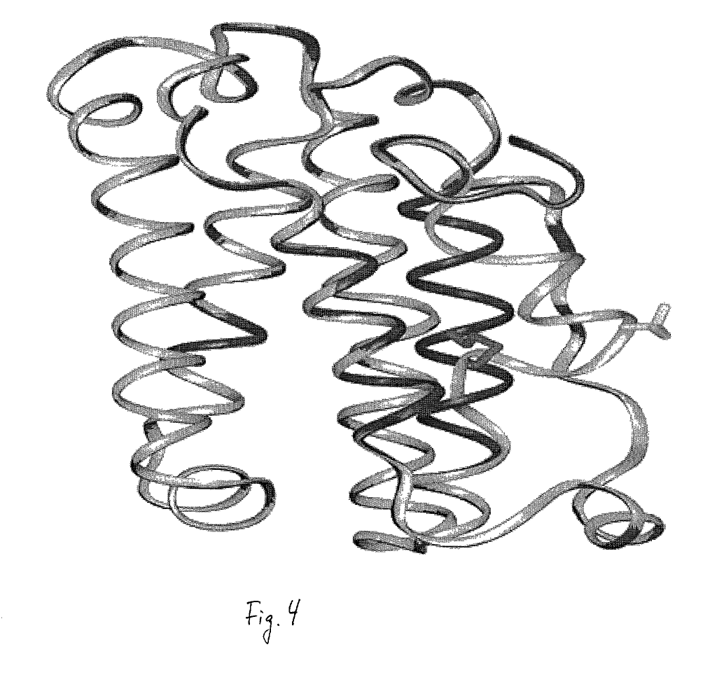

The result of this modeling is visualized in FIG. 4. It indicates that the M148 I mutation, which concerns a residue located in the E loop of IFNα-2, weakly affects the spatial conformation of the E loop and of the A loop which is nearby. The side chains, which are near the position 148 and located on the E and A loops, have a modified orientation. This is particularly true for the R144 residue that is oriented towards the inside of the structure in the wild-type protein and towards the outside in the presence of the M148 I mutation. This change is important since a salt bridge between R144 and E141 is present in the wild-type protein structure. In addition, the side chains of R22 and E141 residues also have a modified orientation in presence of the M148 I mutation. This result suggests that the M148 I (M171I) mutation is a functional SNP that will be confirmed by carrying out biological tests as described below in b).

b) Study of the biological function of M148 I mutant IFNα-2 compared to that of wild-type IFNα-2

(i) Cloning of the wild-type and M148 I mutated mature IFNα-2 in the prokaryotic expression vector pTrc/His-topo:

The nucleotide sequences coding for the wild-type and mutated IFNα-2 protein are as mentioned in the genotyping of M148 I described below.

The PCR products are inserted into the prokaryotic expression vector pTrcHis-topo under the control of the Trc hybrid promoter inducible by IPTG (Iso-Propyl-Thio-Galactoside) by TOPO™-Cloning (Invitrogen Corp.).

This vector enables the heterologous expression of eukaryotic proteins in the bacteria as a result of a minicistronic unit.

The wild-type protein and the mutated protein are produced in the form of fusion proteins carrying an N-terminal extension formed from a 6-histidine tail and the epitope for a specific antibody.

It is possible to cleave this additional region by using the endoprotease Enterokinase.

After verification of the nucleotide sequence in the region of the vector coding for the recombinant proteins, the strain E. coli Top 10 (Invitrogen) is transformed with these recombinant expression vectors.

(ii) Heterologous expression in E. coli and purification of the poly-histidine wild-type and M171 I mutated IFNα-2 fusion proteins:

Two precultures saturated with 100 mL of LBA medium (Luria Bertoni+ampicillin 100 μ/mL) containing a clone coding for the wild-type IFNα-2 and for M171 I mutated IFNα-2 were made overnight at 37° C. with an agitation of 200 rpm, then were used for seeding at 1/10 900 mL of the LBA medium (preincubated overnight at 37° C.).

When this second culture reached a cellular density corresponding to an optical density O.D._{600 nm} of 0.8, the expression of the protein is induced by the addition of IPTG at a final concentration of 1 mM and it is kept for 5 h at 30° C., with an agitation of the culture of 200 rpm.

The pellet of bacteria obtained after centrifugation at 4000×g, 30 min, 4° C., is resuspended in 25 mL of buffer A (Tris 50 mM, pH 8, NaCl 50 mM, imidazole 10 mM, PMSF 0.1 mM pH 8).

Preincubation of 30 min in ice in the presence of 0.5 mg/mL of lysozyme and 20 units of DNase I precedes sonication carried out in three steps with control of temperature of the sample (one step delivered 240 Watt per impulse of 10 sec with 10 sec stop for 1 min). The cell suspension is then clarified by centrifugation at 15,000×g for 30 min at 4° C.

The centrifugation supernatant is next filtered on a 0.22 micrometer-filter.

The poly-histidine proteins present are then purified by HPLC on HlTrap™ Nickel Affinity resin (Amersham Pharmacia Biotech) previously equilibrated in 50 mM Tris, 300 mM NaCl pH 8.0 (Buffer B). After copiously washing the column with 1M NaCl in 50 mM Tris pH 8.0, the elution of the proteins was induced by a linear gradient of imidazole between concentrations of 0.01-0.25 M in buffer B.

The presence of the poly-histidine protein in the collected fractions is verified, on the one hand by SDS PAGE electrophoresis and on the other hand by immunodetection with the aid of a specific antibody directed against the N-terminal end of the fusion protein.

At this stage, the protein of interest is up to 80% pure.

The last step of the purification consists of a separation of the proteins on an ion-exchange chromatography column.

The fractions containing the fusion protein are injected on an anion-exchange column (MiniQ PE 4.6/50, Pharmacia) that was previously equilibrated in 50 mM Tris buffer pH 8. The elution of the proteins is carried out by the passage of an NaCl gradient between 0 and 500 mM in 50 mM Tris buffer pH 8.

The purity of the protein of interest is estimated on the SDS/PAGE gel and the protein concentrations were measured by BCA measurement (bicinchoninic acid and copper sulfate, Sigma).

The purified wild-type and mutated IFNα-2 proteins containing the N-terminal poly-histidine end are used during the functional tests that consist of measurement of the antiproliferative activity of these two forms of IFNα-2 on the growth of the Daudi cell line.

(iii) Evaluation of the ability of wild-type and M148 I mutated IFNα-2 to induce the antiproliferation of the human lymphoblast cells of the Burkitt's Daudi cell line:

These tests are carried out on two different IFNα-2 types, non-mutated IFNα-2 and M148 I IFNα-2 proteins. Cells (human lymphoblasts from the Burkitt's Daudi cell line) previously cultivated in the RPMI 1640 medium (supplemented with 10% fetal bovine serum and 2 mM L-glutamine) are seeded in 96-well plate at a cellular density of 4.10⁴ cells/well.

For each of the IFNs, final concentrations of 0.003 pM to 600 nM are tested. Eight cultures and therefore different measurements are done in parallel for both proteins and for each concentration.

The Daudi cells are then cultivated for 66 hours at 37° C. under 5% CO₂.

After 66 hours of growth the antiproliferative effect of each IFNα-2 is estimated by the number of living cells still presenting mitochondrial dehydrogenase activity. The activity of the dehydrogenase can be detected in the presence of 12 mM MTT (incubated 4 h at 37° C.), by monitoring the optical density at 550 nm corresponding to the formation of formazan crystals derived from cleaving the tetrazolium salt, MTT.

The antiproliferative activity of the wild-type IFNα-2 or M148 I mutated IFNα-2 is based on the measurements of the IC50 corresponding to the concentration of IFNα-2 inhibiting 50% of the cell growth.

The average ratio between the IC50 measured for the mutated IFNα-2 and the IC50 measured for the wild-type IFNα-2 reaches 15.35 (standard deviation 9.35).

Thus, this test shows that the cellular antiproliferative activity is strongly inhibited in the case of M148 I mutated IFNα-2 by comparison with wild-type IFNα-2, demonstrating that the M148 I SNP is functional.

c) Genotyping of the M171 I SNP

A similar method as previously described in the case of genotyping the H57R SNP has been applied for genotyping the g1023a SNP (giving the M171 I SNP on the protein sequence) on a population of individuals chosen substantially at random (provided by Coriell Institute). In a similar manner, with adequate primers, the genotyping has been performed by minisequencing on each nucleotide sequence fragment of the IFNα-2, which has been amplified by PCR from the genomic DNA sequence of each individual of the population.

In this case, the primers were as follows:

Sense primer: gttgtcagagcagaaatcat [SEQ ID NO. 9]

Antisense primer: gttgacaaagaaaaagatct [SEQ ID NO. 10]

The condition retained for the genotyping was:

Sense primer+ddATP-R110+ddGTP-Tamra

Briefly, the results are the following:

on 7 negative controls tested, all were validated and on 239 individuals tested, 238 were genotyped. Thus, the percentage of success of the method of genotyping reaches 99.6%.

Among the 238 genotyped individuals of the random population, only one was heterozygote of the studied SNP. The heterozygote individual was Caribbean.

The allelic frequency and the genotype frequency in the Caribbean population are indicated in the following table:

| | | | | | Geno- | | | | | |
|---|---|---|---|---|---|---|---|---|---|---|
| Total | | Allelic frequency | | | type AA | | Genotype AG | | Genotype GG | |
| N | % | % | 95% | IC 5 | N | % | N | % | N | % |
| 10 | 4.2 | 5.0 | 0.0 | 14.6 | 0 | 0 | 1 | 10.0 | 9 | 90 |

### EXAMPLE 4

### Validation of the Method for Identification of SNPs

The method of SNPs identification, object of the present invention, has been applied to seven known genes arbitrarily chosen because the prior art indicates that SNPs on these genes are involved in various pathogenic states (see following table).

| | | | | SNPs |
|---|---|---|---|---|
| | | | | described in |
| | | Protein | | the prior art |
| | Gene | Accession | | and detected |
| | Accession | Number | | with the |
| | Number | (Swiss- | | present |
| Gene | (GenBank) | Prot) | Pathology | method |
| AGT | AL512328.7 | P01019 | Hypertension | T207M |
| APOE | AF261279 | P02649 | Alzheimer | C130R |
| ADRB2 | J02960 | P07550 | Nocturnal | R16G |
| | | | asthma | |
| | | | Obesity | Q27E |
| COL1A1 | AF017178 | P02452 | Osteoporose | g1546t |
| | | | susceptibility | |
| MTHFR | AC025001 | P42898 | Neural tube | A222V |
| | | | defect | |
| CX26 | AL138688.27 | P29033 | Deafness | 35 del (g), |
| | | | | 167 del (t), |
| | | | | M34T |
| HFE | Z92910.1 | Q30201 | Haemochro- | C282Y |
| | | | matosis | |

The indicated numbers refer to the position on the gene sequence when the nature of the SNP is indicated with small letters and on the protein sequence when the nature of the SNP is indicated with capital letters.

These seven genes are:

the gene encoding for the angiotensin I (AGT), in which the presence of T207M SNPs has been related to hypertension.

the gene encoding for the apolipoprotein E (APOE), in which the presence of C130R SNP has been related to Alzheimer disease.

the gene encoding for the beta-2-adrenergic receptor (ADRB2), in which the presence of R16G SNP has been related to nocturnal asthma and Q27E SNP to obesity.

the gene encoding for the collagen type I, alpha-1 chain (COL1A1), in which the presence of g1546t SNP has been related to osteoporose susceptibility.

the gene encoding for the methylenetetrahydrofolate reductase (MTHFR), in which the presence of A222V SNP has been related to neural tube defect.

the gene encoding for the gap junction protein connexin 26 (CX26), in which the presence of 35del(g), 167del(t), and M34T SNPs has been related to deafness.

the hemochromatosis gene (HFE), in which the presence of C282Y SNP has been related to haemochromatosis.

In the scope of the present invention, a fragment of the nucleotide sequence of each of the seven previously chosen genes, comprising, for example, the coding sequence, was isolated from different individuals in a population of individuals chosen in a random manner (population provided by the Coriell Institute, United States). For each gene, the fragment was isolated by PCR amplification using appropriate sense and antisense primers, as indicated in the following table:

| Gene | Sense primer | Antisense primer |
|---|---|---|
| AGT | ACACAGCTGACAGGCTACAG | GTCACAGCCTGCATGAAC |
| | [SEQ ID NO.11] | [SEQ ID NO.12] |
| APOE | GACGAGACCATGAAGGAGTT | CCGGCCTGGTACACTG |
| | [SEQ ID NO.13] | [SEQ ID NO.14] |
| ADRB2 | AGCCAGTGCGCTTACC | CACATTGCCAAACACGAT |
| | [SEQ ID NO.15] | [SEQ ID NO.16] |
| COL1A1 | TGTCTAGGTGCTGGAGGTTA | GCTTGCGTGGTAGAGACA |
| | [SEQ ID NO.17] | [SEQ ID NO.18] |
| MTHFR | AAGCACTTGAAGGAGAAGGT | AGTTCTGGACCTGAGAGGAG |
| | [SEQ ID NO.19] | [SEQ ID NO.20] |
| CX26 | AAACCGCCCAGAGTAGAA | CCCTTGATGAACTTCCTCTT |
| | [SEQ ID NO.21] | [SEQ ID NO.22] |
| HFECTCCTCATCCTTCCTCTTTCCTCCTGGCTCTCATCAGTC | | |
| [SEQ ID NO. 23] | [SEQ ID NO. 24] | |

Sequencing of each fragment was then carried out on certain of these samples having a heteroduplex profile (that is a profile different from that of their respective reference wild-type gene sequence whose accession number in GenBank database is quoted in the first table given in this example) after analysis by DHPLC (“Denaturing-High Performance Liquid Chromatography”).

The fragments sequenced in this way were then compared to the nucleotide sequence of the fragment of the corresponding reference wild-type gene and the SNPs in conformity with the invention identified.

Thus, the SNPs are natural and each of them is present in certain individuals of the world population.

As indicated in the first table given in this example, the present method allowed the identification and detection of all expected SNPs. Among the detected SNPs according to the invention, seven were coding SNPs (T207M in the AGT gene; C130R in the APOE gene; G16R, Q27E in the ADRB2 gene; A222V in the MTHFR gene; M34T in the CX26 gene; C282Y in the HFE gene), and three were non-coding SNPs (g1546t in the COL1A1 gene; 35del(g), 167del(t) in the CX26 gene).

As a conclusion, these results clearly demonstrate the validity of the method of the invention to detect SNPs, either coding or non-coding, in pathogenic genes involved in several different independent common diseases from the population of individuals chosen substantially at random, without any selection based on any particular phenotype.

1. A method for determining at least one functional SNP in a gene, comprising the following steps:
a) Preselecting a candidate gene;
b) Providing a sample population comprising a significant number of individuals chosen substantially at random from the general population;
c) Isolating from each individual of the sample population at least one fragment of the nucleotide sequence of the preselected candidate gene;
d) Identifying at least one SNP in at least one fragment isolated in step c); and
e) From the SNP(s) identified in step d), identifying those with functionality.

2. The method of claim 1, wherein the significant number of individuals chosen substantially at random in the population in step b) is greater than 100.

3. The method of claim 1, wherein the genotype and/or the phenotype of individuals chosen substantially at random in the population in step b) are not known.

4. The method of claim 1, wherein the fragment of the nucleotide sequence of the candidate gene is isolated in step c) by a PCR or RT-PCR reaction.

5. The method of claim 1, wherein the identification of a SNP in step d) is carried out by at least one method selected from the group consisting of direct sequencing, multiplexing method using denaturing high performance liquid chromatography (DHPLC), single strand conformation polymorphism (SSCP), denaturing gradient gel electrophoresis (DGGE), methods based on the cleavage of the mismatch by chemicals or enzymes, allele-specific hybridization, allele-specific primer extension and allele-specific oligonucleotide ligation.

6. The method according to claim 5, wherein the identification of at least one SNP in step d) is carried out by a multiplexing method using denaturing high performance liquid chromatography (DHPLC).

7. The method according to claim 6, wherein the identification of at least one SNP in step d) is carried out by a multiplexing method using denaturing high performance liquid chromatography (DHPLC) followed by sequencing.

8. The method of claim 5, wherein the determination of the functionality of the SNP in step e) is carried out by at least one method selected among bioinformatic tools such as bioinformatic molecular modeling (in silico) and biological assay (in vivo or in vitro).

9. The method of claim 5, wherein the determination of the functionality of the SNP in step e) is carried out by comparison of functionality between:
i) a wild-type protein encoded by the reference wild-type nucleotide sequence of the preselected candidate gene, and
ii) a mutated protein encoded by a mutated nucleotide sequence of the preselected candidate gene comprising at least one SNP as identified in step d).

10. A method for determining at least one functional SNP in a gene, comprising the following steps:
a) Preselecting a candidate gene;
b) Providing a sample population comprising a significant number of individuals chosen substantially at random from the general population;
c) Isolating from each individual of the sample population at least one fragment of the nucleotide sequence of the preselected candidate gene;
d) Forming one or more mixtures comprising fragments isolated in step c) by randomly mixing fragments from one or more individuals;
e) Conducting an analysis for comparing, between them, the fragments of each mixture formed in step d) in order to determine whether said mixture has a heterozygous or homozygous profile;
f) Forming one or more homogeneous groups comprising at least one mixture analyzed in step e), each of said homogeneous group having an identical heterozygous or homozygous profile;
g) Identifying at least one SNP in:
i) at least one fragment from each homogeneous group having a heterozygous profile formed in step f),
ii) at least one fragment of at least one mixture having an heterozygous profile as determined in step e), and/or
iii) at least one fragment isolated in step c) from an individual incorporated in a mixture having an heterozygous profile as determined in step e);
h) From the SNP(s) identified in step g), identifying those with functionality.

11. The method of claim 10, wherein the analysis conducted in step e) is carried out by a multiplexing method using denaturing high performance liquid chromatography (DHPLC).

12. The method of claim 10, wherein the identification of a SNP in step g) is carried out by sequencing.

13. The method of claim 10, further comprising the step of genotyping all or part of the nucleotide sequence of the preselected candidate gene identified as comprising at least one SNP.

14. The method of claim 13, wherein said genotyping is carried out by minisequencing.

15. A method for the genetic diagnosis of a disease or a resistance to a disease linked to the presence of a mutated nucleotide sequence of the preselected candidate gene in an individual comprising detecting the presence or absence in said individual of at least one functional SNP identified by the method of claim 1.

16. A method for generating a map of genetic markers comprising performing the method of claim 1, on at least one preselected candidate gene.

17. A method of preparing a polynucleotide comprising the nucleotide sequence of the preselected candidate gene comprising at least one functional SNP, comprising the following steps:
a) Determining at least one functional SNP by the method of claim 1; and
b) Producing a polynucleotide comprising a mutated nucleotide sequence of the preselected candidate gene comprising at least one functional SNP determined in step a).

18. A method of preparing a polypeptide comprising an amino acid sequence encoded by the preselected candidate gene comprising at least one coding functional SNP, comprising the following steps:
a) Determining at least one coding functional SNP by the method of claim 1; and
b) Producing a polypeptide comprising a mutated amino acid sequence encoded by the preselected candidate gene comprising at least one coding functional SNP determined in step a).

19. A composition comprising a therapeutically effective amount of a polynucleotide prepared by the method of claim 17, and a pharmaceutically acceptable carrier.

20. A composition comprising a therapeutically effective amount of a polypeptide prepared by the method of claim 18, and a pharmaceutically acceptable carrier.

21. A method for treating an individual having a pathology and/or disease correlated to the presence or absence of a mutated allele comprising at least one functional SNP in a gene linked to said pathology and/or disease comprising administering a therapeutically effective amount of a polynucleotide prepared according to claim 17 and/or polypeptide prepared according to claim 18 and a pharmaceutically acceptable carrier.

22. A databank comprising functional SNPs determined by the method of claim 1.

23. A method for creating a databank of functional SNPs comprising performing the method according to claim 1, for at least one preselected candidate gene, and collecting said functional SNPs identified by said method.

24. A method for identifying the functional SNP(s) associated with at least one pathology and/or disease or the resistance thereto, comprising analyzing the databank of 22 for statistically relevant associations.


SEQUENCE LISTING (24 sequences)

SEQ ID NO: 1 - DNA, 20 bp, Homo sapiens
gcctcttatgtacccacaaa

SEQ ID NO: 2 - DNA, 20 bp, Homo sapiens
caccagtaaagcaaaggtca

SEQ ID NO: 3 - DNA, 20 bp, Homo sapiens
cacccatttcaaccagtcta

SEQ ID NO: 4 - DNA, 19 bp, Homo sapiens
agctggcatacgaatcaat

SEQ ID NO: 5 - DNA, 535 bp, Homo sapiens
gcctcttatgtacccacaaaaatctattttcaaaaaagttgctctaagaatatagttatcaagttaagtaaaatgtcaatagccttttaatttaatttttaattgttttatcattctttgcaataataaaacattaactttatactttttaatttaatgtatagaatagagatatacataggatatgtaaatagatacacagtgtatatgtgattaaaatataatgggagattcaatcagaaaaaagtttctaaaaaggctctggggtaaaagaggaaggaaacaataatgaaaaaaatgtggtgagaaaaacagctgaaaacccatgtaaagagtgtataaagaaagcaaaaagagaagtagaaagtaacacaggggcatttggaaaatgtaaacgagtatgttccctatttaaggctaggcacaaagcaaggtcttcagagaacctggagcctaaggtttaggctcacccatttcaaccagtctagcagcatctgcaacatctacaatggccttgacctttgctttactggtg

SEQ ID NO: 6 - DNA, 655 bp, Homo sapiens
cacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattgattcgtatgccagct

SEQ ID NO: 7 - DNA, 20 bp, Homo sapiens
ctcctgcttgaaggacagac

SEQ ID NO: 8 - DNA, 20 bp, Homo sapiens
cctggggaaatccaaagtca

SEQ ID NO: 9 - DNA, 20 bp, Homo sapiens
gttgtcagagcagaaatcat

SEQ ID NO: 10 - DNA, 20 bp, Homo sapiens
gttgacaaagaaaaagatct

SEQ ID NO: 11 - DNA, 20 bp, Homo sapiens
acacagctgacaggctacag

SEQ ID NO: 12 - DNA, 18 bp, Homo sapiens
gtcacagcctgcatgaac

SEQ ID NO: 13 - DNA, 20 bp, Homo sapiens
gacgagaccatgaaggagtt

SEQ ID NO: 14 - DNA, 16 bp, Homo sapiens
ccggcctggtacactg

SEQ ID NO: 15 - DNA, 16 bp, Homo sapiens
agccagtgcgcttacc

SEQ ID NO: 16 - DNA, 18 bp, Homo sapiens
cacattgccaaacacgat

SEQ ID NO: 17 - DNA, 20 bp, Homo sapiens
tgtctaggtgctggaggtta

SEQ ID NO: 18 - DNA, 18 bp, Homo sapiens
gcttgcgtggtagagaca

SEQ ID NO: 19 - DNA, 20 bp, Homo sapiens
aagcacttgaaggagaaggt

SEQ ID NO: 20 - DNA, 20 bp, Homo sapiens
agttctggacctgagaggag

SEQ ID NO: 21 - DNA, 18 bp, Homo sapiens
aaaccgcccagagtagaa

SEQ ID NO: 22 - DNA, 20 bp, Homo sapiens
cccttgatgaacttcctctt

SEQ ID NO: 23 - DNA, 20 bp, Homo sapiens
ctcctcatccttcctctttc

SEQ ID NO: 24 - DNA, 19 bp, Homo sapiens
ctcctggctctcatcagtc